(19) United States
(12) Patent Application Publication
(10) Pub. No.: US 2020/0239622 A1
(43) Pub. Date: Jul. 30, 2020

(54) DYNAMIC UREA BONDS FOR POLYMERS

(71) Applicant: THE BOARD OF TRUSTEES OF THE UNIVERSITY OF ILLINOIS, Urbana, IL (US)

(72) Inventors: Jianjun Cheng, Champaign, IL (US); Hanze Ying, Champaign, IL (US); Yanfeng Zhang, Urbana, IL (US)

(21) Appl. No.: 16/844,436

(22) Filed: Apr. 9, 2020

Related U.S. Application Data
(62) Division of application No. 15/519,853, filed on Apr. 18, 2017.
(60) Provisional application No. 62/069,384, filed on Oct. 28, 2014.
(60) Provisional application No. 62/069,385, filed on Oct. 28, 2014.

(51) Int. Cl. C08G 18/79; C08G 18/81; C08G 18/76; C08G 18/32; C08G 18/67; A61L 27/18; A61L 17/10; A61K 47/34; C08G 18/24; C08G 18/75

(52) U.S. Cl. CPC C08G 18/792; C08G 18/8175; C08G 18/765; C08G 18/325; C08G 18/6795; A61L 27/18; C08G 2280/00; A61K 47/34; C08G 18/3271; C08G 18/246; C08G 18/7642; C08G 18/757; A61L 17/10

(57) ABSTRACT

The present invention relates to polymers having dynamic urea bonds and more specifically to polymers having hindered urea bonds (HUBs). The present invention also relates to: (a) malleable, repairable, and reprogrammable shape memory polymers having HUBs, (b) reversible or degradable (e.g., via hydrolysis or aminolysis) linear, branched or network polymers having HUBs, and (c) to precursors for incorporation of HUBs into these polymers. The HUB technology can be applied to and integrated into a variety of polymers, such as polyureas, polyurethanes, polyesters, polyamides, polycarbonates, polyamines, and polysaccharides to make linear, branched, and cross-linked polymers. Polymers incorporating the HUBs can be used in a wide variety of applications including plastics, coatings, adhesives, biomedical applications, such as drug delivery systems and tissue engineering, environmentally compatible packaging materials, and 4D printing applications.

### FEDERAL FUNDING LEGEND

This invention was made with government support under Grant No. CHE1153122 awarded by the United States National Science Foundation and the Director's New Innovator Award 1DP2OD007246-01 awarded by the United States National Institute of Health. The government has certain rights in the invention.

### FIELD OF THE INVENTION

The present invention relates to polymers having dynamic bonds such as dynamic urea bonds and more specifically to polymers having hindered urea bonds (HUBs). The present invention also relates to: (a) malleable, repairable, and reprogrammable shape memory polymers having HUBs, (b) reversible or degradable (e.g., via hydrolysis or aminolysis) linear, branched or network polymers having HUBs, and (c) to precursors for incorporation of HUBs into these polymers. The HUB technology can be applied to and integrated into a variety of polymers, such as polyureas, polyurethanes, polyesters, polyamides, polycarbonates, polyamines, and polysaccharides to make linear, branched, and cross-linked polymers. Polymers incorporating the HUBs can be used in a wide variety of applications including plastics, coatings, adhesives, biomedical applications, such as drug delivery systems and tissue engineering, environmentally compatible packaging materials, and 4D printing applications.

### BACKGROUND OF THE INVENTION

There is a need in the material and polymer sciences to develop polymeric materials with desired in-use performance characteristics that are also malleable, repairable, and shape reprogrammable. There is also a need to develop such polymers that can be degraded or reversibly depolymerized. Even though shape memory and self-healing polymers are known, many of these polymers do not have both the desired performance and dynamic characteristics. For example, many shape memory polymers, which depend on the formation of covalent cross-links, cannot be processed, reprogrammed, or recycled after the permanent shape is set by covalent crosslinking. With respect to degradable or reversibly depolymerizable polymers, these polymers often lack the required in-use performance characteristics and are either too easily degraded or on the other hand not degraded as readily or rapidly as desired.

Differing from polymers formed with strong, irreversible covalent bonds and stable bulk properties, polymers prepared through reversible non-covalent interactions or covalent bonds exhibit various dynamic properties. The dynamic features of reversible polymers have been employed in the design of self-healing, shape-memory, and environmentally adaptive materials. However, non-covalent interactions are relatively weak, with only a few exceptions such as quadruple hydrogen bonding, high valence metal chelation, and host-guest molecular interactions. Dynamic covalent bonds, on the contrary, usually have higher strength and more controllable reversibility.

The amide bond forms the basic structure of numerous biological and commodity polymers, for example nylon and polypeptides, and as such, is one of the most important organic functional groups. It has been hypothesized that the amide bond has relatively high stability due to conjugation effects between the lone electron pair on the nitrogen atom and the pi-electrons on the carbonyl p-orbital. Reversing the amide bond, i.e. amidolysis, usually requires extreme conditions, such as highly basic or acidic conditions and/or high temperatures, or the presence of special reagents, such as catalysts and enzymes.

Introducing bulky substituents has been theorized to create steric hindrance to disturb the orbital co-planarity of the amide bond, which diminishes the conjugation effect and thus weakens the carbonyl-amine interaction. However, the dissociated intermediate from amidolysis, would be a ketene, and if formed would generally be too reactive to provide dynamic reversible formation of the amide bond. To make the carbonyl-amine structure reversible, it is required that the dissociated carbonyl structure be stable under ambient conditions but still highly reactive with amines. One such functional group that satisfies these requirements is the isocyanate group, which can be used to form urea linkages. Isocyanates are generally sufficiently stable under ambient conditions and can react with amines rapidly to form a urea bond, a reaction that has been broadly used in the synthesis of polyureas and poly(urethane-ureas). Therefore, it would be highly desirable to control the reversibility and the kinetics of these urea bonds in polymeric materials.

Many currently available polymeric materials lack both the desired performance characteristics and dynamic properties, as it is difficult to achieve both these properties from conventional polymer technologies. For example, highly covalent cross-linked network polymers generally lack the ability to be recycled, processed and self-healed after cracks have developed. As another example, polyureas constitute an important class of polymers, however, polyureas generally have a very stable bond, are not very soluble, and cannot be recycled and reshaped after polymerization.

There is also a need to develop high performance polymers for biomedical applications including drug delivery systems, scaffolds for tissue regeneration, surgical sutures, and transient medical devices and implants, which usually require short functioning times and complete degradation and clearance after use. Also such polymers would be useful for controlled release systems in agroindustry and for degradable, environmentally friendly plastics and packaging materials. Polyesters are the most widely used, conventional hydrolysable materials. A large variety of other hydroly sable polymers bearing orthoester, acetal, ketal, aminal, hemiaminal, imine, phosphoester, and phosphazene bonds have also been reported. However, many of these hydrolysable polymers do not have the desired balance of performance characteristics and degradation kinetics

Also, with the growing importance of 3D printing technologies there is a need for the development of polymeric materials that can be used in such applications. However, once the product is produced with a 3D printer, the product often lacks so-called 4D characteristics, i.e. where the product can be further processed, manipulated, or shaped. Many polymeric materials used in 3D printing lack this further 4D characteristic.

Separate from these challenges there is an overarching question of sustainability and environmental stewardship in the production and use of products. It would be highly desirable to develop polymeric materials having the desired in-use performance characteristics that are biodegradable or that can be readily recycled.

See H. Ying et al, Dynamic urea bond for the design of reversible and self-healing polymers, J. Nature Communications, 5, 3218,published Feb. 5, 2014, and PCT Publication WO 2014/144539 A2, to The Board of Trustees of the University of Illinois, published Sep. 18, 2014, which are both incorporated by reference herein in their entirety.

As seen from the foregoing, it would be highly desirable to have improved polymers. It is apparent there is an ongoing need to develop new polymers that have both desired and controlled dynamic characteristics without compromising other in-use performance properties.

We have surprisingly found that HUBs can be used to prepare malleable, repairable, and reprogrammable shape memory polymers, as well as reversible or degradable polymers, such as water degradable or hydrolysable polymers. We have also surprisingly found that HUBs can be incorporated into a range of precursors to provide an efficient and flexible means for making these polymers, because the desired polymers can be synthesized from the precursor monomers by simple combination and generally without the need for a catalyst.

### BRIEF DESCRIPTION OF THE FIGURES

FIG. 1 depicts the shape memory process for the shape memory polymers (SMPs) of the present invention. The polymeric material starts out with a Permanent Shape that is in a rigid form (box at left). When the polymer is heated above Tg (the glass transition temperature), the material becomes flexible and stretchable and is in its Flexible Shape (box at top). Cooling the polymeric material back below its Tg will bring it back to its Permanent Shape. When in the Flexible Shape, if an external force is applied, the material will deform and can be Shape Reprogrammed (box at right). If the temperature is then brought down below the Tg while the force is still applied, the material will transform into a temporary shape, which is also fixed or rigid, the Temporary Shape Fixed (box at bottom), but has a different shape from the initial state (i.e. the permanent shape). If the force is then removed and the material is reheated above the Tg, the material will go back to its Flexible Shape. It should be noted that when the material is above the Tg the material will be in the flexible state and have a Flexible Shape, but the shape will be the same as that of the Permanent Shape.

FIG. 2 depicts a dog bone shaped polymeric material made with a HUB polymer. As the dog bone is pulled apart or cut it is seen from the exploded views that the HUBs of the polymer can dissociate. These bonds can then re-associate to heal or reform the dog bone.

FIG. 3 is an illustration of the hydrolysis mechanism of hindered urea bonds (HUBs). The urea bond is destabilized by bulky substituent induced bond rotation and a loss of conjugation effect. Also, with respect to this FIG. 3, R₁ and R₂ are independently selected from the group consisting of (C₁-C₂₀)alkyl, (C₄-C₁₀)cyclolalkyl, (C₁-C₂₀)alkyl(C₄-C₁₀)cycloalkyl, (C₁-C₂₀)alkyl(C₄-C₁₀)cycloalkyl(C₁-C₂₀)alkyl, C₂-C₂₀)alkyl-PEG-(C₂-C₂₀)alkyl, and H, and combinations thereof.

FIGS. 4A through 4D depict the dynamicity and hydrolytic degradation of HUB-containing model compounds: FIG. 4A: Parameters related to the hydrolytic degradation of HUBs; FIG. 4B: Structures of five HUB-containing model compounds; FIG. 4C Binding constants (Keq), dissociation rate (k-1) and water degradation kinetics of the five HUB-containing model compounds shown in FIG. 4B; and FIG. 4D: Representative NMR spectra showing the degradation of compound 3 of FIG. 4B. The percentage of hydrolysis was determined by the integral ratio of peaks corresponding to starting compounds and hydrolysis products as shown in the inset.

FIGS. 5A through 5C depict the water degradation of HUB based linear polymers (pHUBs), or polymeric HUBs: FIG. 5A: Shows the synthesis of four different types of pHUBs by mixing diisocyanates and diamines; FIG. 5B: GPC curves showing water degradation of poly(6/9) and poly(7/9) in H₂O/DMF=5:95 after 24 h incubation at 37° C.; and FIG. 5C: Plot showing molecular weight reduction of the four polymers depicted in FIG. 5A in H₂O/DMF=5:95 for various incubation times at 37° C.

FIGS. 6A through 6D depict the water degradation of HUB based cross-linked polymers (pHUBs). FIG. 6A: Triisocyanate and diamine cross-linked into an organogel in DMF with the pre-addition of water; FIG. 6B: Synthesis of urea based cross-linked hydrophilic polymers G1, G2, and G3 by UV polymerization; FIG. 6C: Organo-gel synthesized from material of FIG. 6A collapsed into solution after 24 h incubation at 37° C. FIG. 6D: Weight change of G1, G2, and G3 after immersing in phosphate-buffered saline (PBS) for variant times.

### SUMMARY OF THE INVENTION

The present invention relates to polymers having dynamic bonds such as dynamic urea bonds and more specifically to polymers having hindered urea bonds (HUBs). The present invention also relates to: (a) malleable, repairable, and reprogrammable shape memory polymers having HUBs, (b) reversible or degradable (e.g., via hydrolysis or aminolysis) linear, branched or network polymers having HUBs, and (c) to precursors for incorporation of HUBs into these polymers. The HUB technology can be applied to and integrated into a variety of polymers, such as polyureas, polyurethanes, polyesters, polyamides, polycarbonates, polyamines, and polysaccharides to make linear, branched, and cross-linked polymers. Polymers incorporating the HUBs can be used in a wide variety of applications including plastics, coatings, adhesives, biomedical applications, such as drug delivery systems and tissue engineering, environmentally compatible packaging materials, and 4D printing applications.

### DETAILED DESCRIPTION OF THE INVENTION

The present invention relates to a hindered urea bond polymer comprising recurring units from: (a) a hindered amine substituted monomer, and (b) a crosslinking agent substituted with two or more isocyanate groups.

In one aspect the present invention relates to a hindered urea bond polymer comprising the reaction product from: (a) a hindered amine substituted monomer, and (b) a crosslinking agent substituted with two or more isocyanate groups.

In another aspect the present invention relates to a polymer wherein the hindered amine-substituted monomer is selected from acrylates, butadienes, ethylenes, norbornenes, styrenes, vinyl chlorides, vinyl esters, vinyl ethers, and combinations thereof.

In another aspect the present invention relates to a hindered amine substituted monomer such that the amino function is not directly attached to an aromatic group. In other words it is not an aromatic amine.

In another aspect the present invention relates to a polymer wherein the hindered amine-substituted monomer is selected from

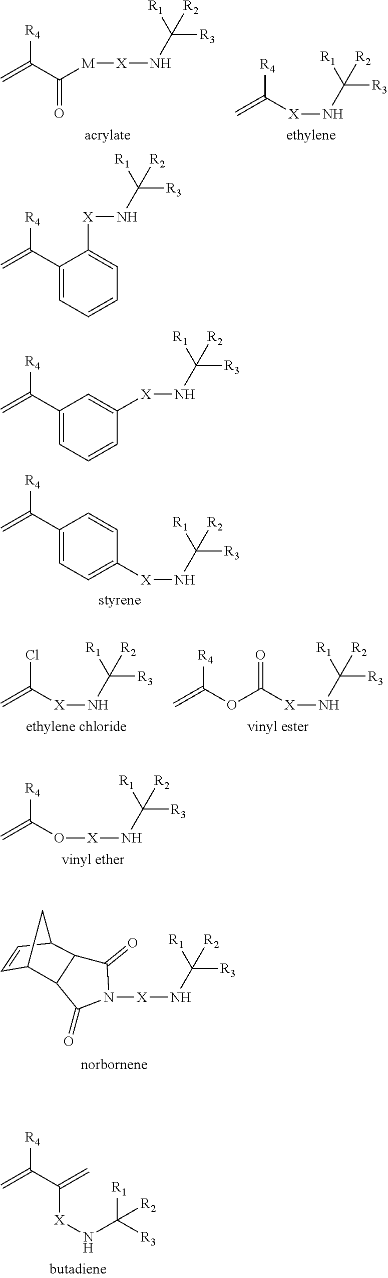

and combinations thereof, wherein R₁, R₂, R₃, and R₄ are independently selected from the group consisting of (C₁-C₂₀)alkyl, (C₄-C₁₀)cyclolalkyl, (C₁-C₂₀)alkyl(C₄-C₁₀)cycloalkyl, (C₁-C₂₀)alkyl(C₄-C₁₀)cycloalkyl(C₁-C₂₀)alkyl, (C₁-C₂₀)alkyl(C₆-C₁₀)aryl(C₁-C₂₀)alkyl, (C₂-C₂₀)alkyl-PEG-(C₂-C₂₀)alkyl, and H, and combinations thereof; and M and X are independently selected from a single bond, (C₁-C₂₀)alkyl, (C₄-C₁₀)cyclolalkyl, (C₁-C₂₀)alkyl(C₄-C₁₀)cycloalkyl, (C₁-C₂₀)alkyl(C₄-C₁₀)cycloalkyl(C₁-C₂₀)alkyl, (C₁-C₂₀)alkyl(C₆-C₁₀)aryl(C₁-C₂₀)alkyl, and (C₂-C₂₀)alkyl-PEG-(C₂-C₂₀)alkyl, and combinations thereof, wherein X is not a single bond when attached to an aromatic ring as in, for example, the three styrene structures.

In another aspect the present invention relates to a polymer wherein R₁, R₂, R₃, are each methyl, R₄ is selected from H, methyl, and ethyl.

In another aspect the present invention relates to a polymer wherein R₄ is selected from H and methyl.

In another aspect the present invention relates to a polymer wherein R₄ is H.

In another aspect the present invention relates to a polymer wherein the crosslinking agent is OCN—Y—NCO, where Y is selected from (C₂-C₂₀)alkyl, (C₄-C₁₀)cyclolalkyl, (C₁-C₂₀)alkyl(C₄-C₁₀)cycloalkyl, (C₁-C₂₀)alkyl(C₄-C₁₀)cycloalkyl(C₁-C₂₀)alkyl, (C₁-C₂₀)alkyl(C₆-C₁₀)aryl(C₁-C₂₀)alkyl, and (C₂-C₂₀)alkyl-PEG-(C₂-C₂₀)alkyl, and combinations thereof.

In another aspect the present invention relates to a crosslinking agent such that the isocyanate function is not directly attached to an aromatic group. In other words it is not an aromatic isocyanate.

In another aspect the present invention relates to a hindered urea bond polymer made by a process comprising: (a) reacting a hindered amine substituted monomer, and (b) a crosslinking agent substituted with two or more isocyanate groups.

In another aspect the present invention relates to a hindered urea bond polymer comprising recurring units from (a) an isocyanate-substituted monomer, and (b) a crosslinking agent substituted with two or more hindered amine groups.

In another aspect the present invention relates to a hindered urea bond polymer comprising the reaction product from (a) an isocyanate-substituted monomer, and (b) a crosslinking agent substituted with two or more hindered amine groups.

In another aspect the present invention relates to a polymer wherein the isocyanate-substituted monomer is selected from acrylates, butadienes, ethylenes, norbornenes, styrenes, vinyl chlorides, vinyl esters, vinyl ethers, and combinations thereof.

In another aspect the present invention relates to an isocyanate-substituted monomer selected from acrylates, butadienes, ethylenes, norbornenes, styrenes, vinyl chlorides, vinyl esters, vinyl ethers, and combinations thereof.

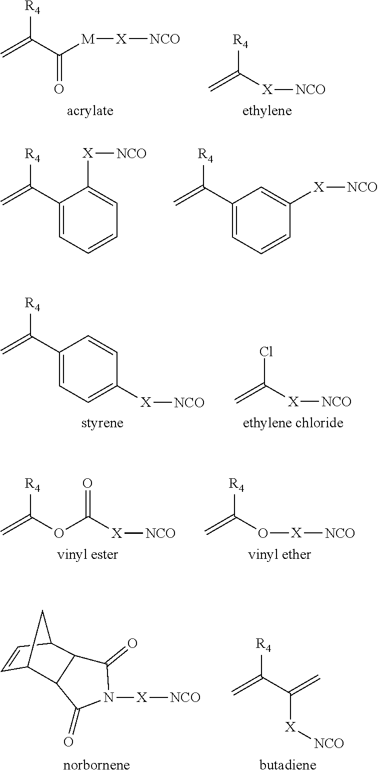

wherein R₄ is selected from the group consisting of (C₁-C₂₀)alkyl, (C₄-C₁₀)cyclolalkyl, (C₁-C₂₀)alkyl(C₄-C₁₀)cycloalkyl, (C₁-C₂₀)alkyl(C₄-C₁₀)cycloalkyl(C₁-C₂₀)alkyl, (C₁-C₂₀)alkyl(C₆-C₁₀)aryl(C₁-C₂₀)alkyl, (C₂-C₂₀)alkyl-PEG-(C₂-C₂₀)alkyl, and H, and combinations thereof; and M and X are independently selected from a single bond, (C₁-C₂₀)alkyl, (C₄-C₁₀)cyclolalkyl, (C₁-C₂₀)alkyl(C₄-C₁₀)cycloalkyl, (C₁-C₂₀)alkyl(C₄-C₁₀)cycloalkyl(C₁-C₂₀)alkyl, (C₁-C₂₀)alkyl(C₆-C₁₀)aryl(C₁-C₂₀)alkyl, and (C₂-C₂₀)alkyl-PEG-(C₂-C₂₀)alkyl, and combinations thereof, wherein X is not a single bond when attached to an aromatic ring as in, for example, the three styrene structures.

In another aspect the present invention relates to a polymer wherein the crosslinking agent is

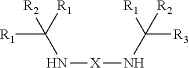

wherein R₁, R₂, and R₃, are independently selected from the group consisting of (C₁-C₂₀)alkyl, (C₄-C₁₀)cyclolalkyl, (C₁-C₂₀)alkyl(C₄-C₁₀)cycloalkyl, (C₁-C₂₀)alkyl(C₄-C₁₀)cycloalkyl(C₁-C₂₀)alkyl, (C₁-C₂₀)alkyl(C₆-C₁₀)aryl(C₁-C₂₀)alkyl, (C₂-C₂₀)alkyl-PEG-(C₂-C₂₀)alkyl, and H, and combinations thereof; and X is selected from (C₂-C₂₀)alkyl, (C₄-C₁₀)cyclolalkyl, (C₁-C₂₀)alkyl(C₄-C₁₀)cycloalkyl, (C₁-C₂₀)alkyl(C₄-C₁₀)cycloalkyl(C₁-C₂₀)alkyl, (C₁-C₂₀)alkyl(C₆-C₁₀)aryl(C₁-C₂₀)alkyl, and (C₂-C₂₀)alkyl-PEG-(C₂-C₂₀)alkyl, and combinations thereof. It is to be noted in this instance that the two nitrogen atoms are to be separated by at least 2 carbon atoms.

In another aspect the present invention relates to a crosslinking agent such that the amine functions are not directly attached to an aromatic group. In other words it is not an aromatic amine.

In another aspect the present invention relates to a polymer wherein for the crosslinking agent, R₁, R₂, R₃, are each methyl.

In another aspect the present invention relates to a hindered urea bond polymer made by a process comprising; (a) reacting an isocyanate-substituted monomer, and (b) a crosslinking agent substituted with two or more hindered amine groups.

In another aspect the present invention relates to a hindered urea bond polymer comprising recurring units from: (a) a hindered amine substituted monomer selected from hindered amine-substituted hydroxyl acids, hindered amine substituted amino acids, and hindered amine substituted epoxides, and (b) a crosslinking agent substituted with two or more isocyanate groups.

In another aspect the present invention relates to a hindered urea bond polymer comprising the reaction product from: (a) a hindered amine substituted monomer selected from hindered amine-substituted hydroxyl acids, hindered amine substituted amino acids, and hindered amine substituted epoxides, and (b) a crosslinking agent substituted with two or more isocyanate groups.

In another aspect the present invention relates to a polymer wherein the hindered amine-substituted monomer is selected from

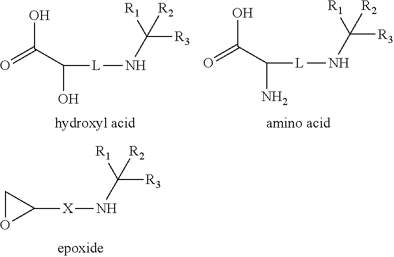

and combinations thereof, wherein R₁, R₂, R₃, are independently selected from the group consisting of (C₁-C₂₀)alkyl, (C₄-C₁₀)cyclolalkyl, (C₁-C₂₀)alkyl(C₄-C₁₀)cycloalkyl, (C₁-C₂₀)alkyl(C₄-C₁₀)cycloalkyl(C₁-C₂₀)alkyl, (C₁-C₂₀)alkyl(C₆-C₁₀)aryl(C₁-C₂₀)alkyl, (C₂-C₂₀)alkyl-PEG-(C₂-C₂₀)alkyl, and H, and combinations thereof; and X and L are independently selected from a single bond, (C₁-C₂₀)alkyl, (C₄-C₁₀)cyclolalkyl, (C₁-C₂₀)alkyl(C₄-C₁₀)cycloalkyl, (C₁-C₂₀)alkyl(C₄-C₁₀)cycloalkyl(C₁-C₂₀)alkyl, (C₁-C₂₀)alkyl(C₆-C₁₀)aryl(C₁-C₂₀)alkyl, and (C₂-C₂₀)alkyl-PEG-(C₂-C₂₀)alkyl, and combinations thereof.

In another aspect the present invention relates to a polymer wherein R₁, R₂, R₃, are each methyl.

In another aspect the present invention relates to a polymer wherein the crosslinking agent is OCN—X—NCO, where X is selected from (C₂-C₂₀)alkyl, (C₄C₁₀)cyclolalkyl, (C₁-C₂₀)alkyl(C₄-C₁₀)cycloalkyl, (C₁-C₂₀)alkyl(C₄-C₁₀)cycloalkyl(C₁-C₂₀)alkyl, (C₁-C₂₀)alkyl(C₆-C₁₀)aryl(C₁-C₂₀)alkyl, and (C₂-C₂₀)alkyl-PEG-(C₂-C₂₀)alkyl, and combinations thereof.

In another aspect the present invention relates to a polymer wherein when the hindered amine monomer is an epoxide, the polymer further comprises recurring units selected from a multi-arm amine.

In another aspect the present invention relates to a hindered urea bond polymer made by a process comprising: (a) reacting a hindered amine substituted monomer in a condensation polymerization reaction, and (b) then reacting the resulting condensation polymer with a crosslinking agent substituted with two or more isocyanate groups.

In another aspect the present invention relates to a hindered amine monomeric precursor selected from the group consisting of:

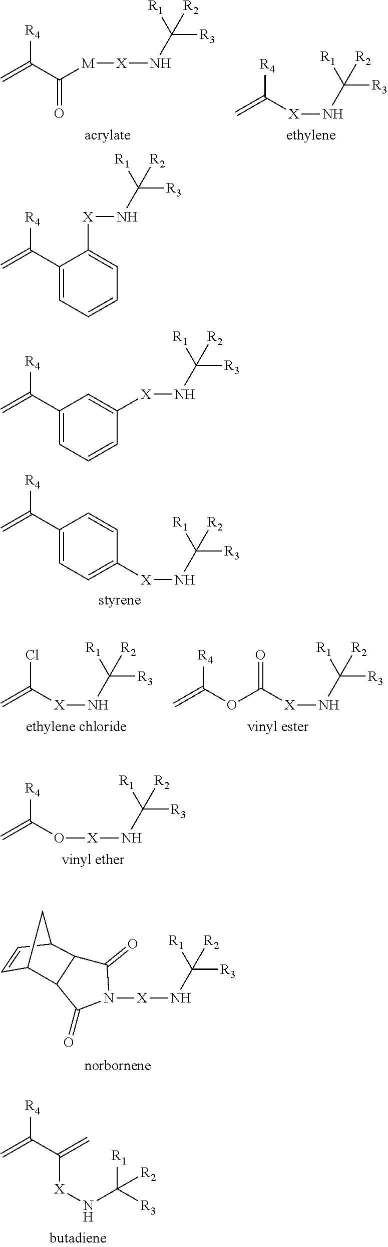

and combinations thereof, wherein R₁, R₂, R₃, and R₄ are independently selected from the group consisting of (C₁-C₂₀)alkyl, (C₄-C₁₀)cyclolalkyl, (C₁-C₂₀)alkyl(C₄-C₁₀)cycloalkyl, (C₁-C₂₀)alkyl(C₄-C₁₀)cycloalkyl(C₁-C₂₀)alkyl, (C₁-C₂₀)alkyl(C₆-C₁₀)aryl(C₁-C₂₀)alkyl, (C₂-C₂₀)alkyl-PEG-(C₂-C₂₀)alkyl, and H, and combinations thereof; and M and X are independently selected from a single bond, (C₁-C₂₀)alkyl, (C₄-C₁₀)cyclolalkyl, (C₁-C₂₀)alkyl(C₄-C₁₀)cycloalkyl, (C₁-C₂₀)alkyl(C₄-C₁₀)cycloalkyl(C₁-C₂₀)alkyl, (C₁-C₂₀)alkyl(C₆-C₁₀)aryl(C₁-C₂₀)alkyl, and (C₂-C₂₀)alkyl-PEG-(C₂-C₂₀)alkyl, and combinations thereof, wherein X is not a single bond when attached to an aromatic ring as in, for example, the three styrene structures.

In another aspect the present invention relates to a highly cross-linked polymer comprising a hindered bond functional group corresponding to the following formula (I)

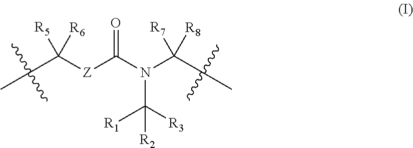

wherein X is O or S; Z is O, S, or NR₄; and R₁, R₂, R₃, R₄, R₅, R₆, R₇, and R₈ are independently selected from the group consisting of (C₁-C₂₀)alkyl, (C₄-C₁₀)cyclolalkyl, (C₁-C₂₀)alkyl(C₄-C₁₀)cycloalkyl, (C₁-C₂₀)alkyl(C₄-C₁₀)cycloalkyl(C₁-C₂₀)alkyl, (C₁-C₂₀)alkyl(C₆-C₁₀)aryl(C₁-C₂₀)alkyl, (C₂-C₂₀)alkyl-PEG-(C₂-C₂₀)alkyl, and H, and combinations thereof.

In another aspect the present invention relates to a highly crosslinked polymer wherein X is O.

In another aspect the present invention relates to a highly crosslinked polymer wherein Z is NR₄.

In another aspect the present invention relates to a highly crosslinked polymer wherein R₁, R², R₃, are each methyl.

In another aspect the present invention relates to a highly crosslinked polymer according wherein R₄ is selected from H and methyl.

In another aspect the present invention relates to a highly crosslinked polymer wherein R₄ is H.

In another aspect the present invention relates to a hydrolysable, malleable, or reprogrammable polymer comprising a hindered bond functional group corresponding to the following formula (I)

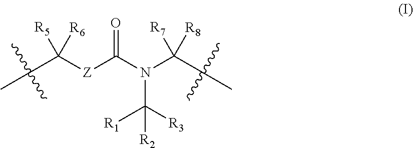

wherein X is O or S; Z is O, S, or NR₄; and R₁, R₂, R₃, R₄, R₅, R₆, R₇, and R₈ are independently selected from the group consisting of (C₁-C₂₀)alkyl, (C₄-C₁₀)cyclolalkyl, (C₁-C₂₀)alkyl(C₄-C₁₀)cycloalkyl, (C₁-C₂₀)alkyl(C₄-C₁₀)cycloalkyl(C₁-C₂₀)alkyl, (C₁-C₂₀)alkyl(C₆-C₁₀)aryl(C₁-C₂₀)alkyl, (C₂-C₂₀)alkyl-PEG-(C₂-C₂₀)alkyl, and H, and combinations thereof.

In another aspect the present invention relates to a hydrolysable, malleable, or reprogrammable polymer wherein X is O.

In another aspect the present invention relates to a hydrolysable, malleable, or reprogrammable polymer wherein Z is NR4.

In another aspect the present invention relates to a hydrolysable, malleable, or reprogrammable polymer wherein R₁, R₂, R₃, are each methyl.

In another aspect the present invention relates to a hydrolysable, malleable, or reprogrammable polymer wherein R₄ is selected from H and methyl.

In another aspect the present invention relates to a hydrolysable, malleable, or reprogrammable polymer wherein R₄ is H.

In another aspect the present invention relates to a malleable polymer comprising a hindered bond functional group corresponding to the following formula (I)

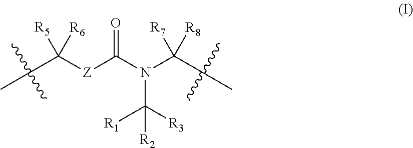

wherein X is O or S; Z is O, S, or NR₄; and R₁, R₂, R₃, R₄, R₅, R₆, R₇, and R₈ are independently selected from the group consisting of (C₁-C₂₀)alkyl, (C₄-C₁₀)cyclolalkyl, (C₁-C₂₀)alkyl(C₄-C₁₀)cycloalkyl, (C₁-C₂₀)alkyl(C₄-C₁₀)cycloalkyl(C₁-C₂₀)alkyl, (C₁-C₂₀)alkyl(C₆-C₁₀)aryl(C₁-C₂₀)alkyl, (C₂-C₂₀)alkyl-PEG-(C₂-C₂₀)alkyl, and H, and combinations thereof.

In another aspect the present invention relates to a reprogrammable polymer comprising a hindered bond functional group corresponding to the following formula (I)

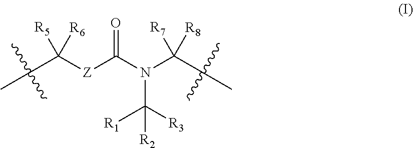

wherein X is O or S; Z is O, S, or NR₄; and R₁, R₂, R₃, R₄, R₅, R₆, R₇, and R₈ are independently selected from the group consisting of (C₁-C₂₀)alkyl, (C₄-C₁₀)cyclolalkyl, (C₁-C₂₀)alkyl(C₄-C₁₀)cycloalkyl, (C₁-C₂₀)alkyl(C₄-C₁₀)cycloalkyl(C₁-C₂₀)alkyl, (C₁-C₂₀)alkyl(C₆-C₁₀)aryl(C₁-C₂₀)alkyl, (C₂-C₂₀)alkyl-PEG-(C₂-C₂₀)alkyl, and H, and combinations thereof; and wherein said polymer has a glass transition between about 20° C. and about 100° C.

In another aspect the present invention relates to a hydrolysable polymer comprising a hindered bond functional group.

In another aspect the present invention relates to a hydrolysable polymer comprising a hindered bond functional group corresponding to the following formula (I)

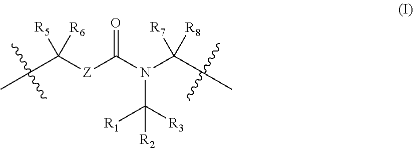

wherein X is O or S; Z is O, S, or NR₄; and R₁, R₂, R₃, R₄, R₅, R₆, R₇, and R₈ are independently selected from the group consisting of (C₁-C₂₀)alkyl, (C₄-C₁₀)cyclolalkyl, (C₁-C₂₀)alkyl(C₄-C₁₀)cycloalkyl, (C₁-C₂₀)alkyl(C₄-C₁₀)cycloalkyl(C₁-C₂₀)alkyl, (C₁-C₂₀)alkyl(C₆-C₁₀)aryl(C₁-C₂₀)alkyl, (C₂-C₂₀)alkyl-PEG-(C₂-C₂₀)alkyl, and H, and combinations thereof.

In another aspect the present invention relates to a hydrolysable polymer wherein X is O.

In another aspect the present invention relates to a hydrolysable polymer according to wherein R₁, R₂, R₃, are each methyl.

In another aspect the present invention relates to a hydrolysable polymer wherein Z is NR₄.

In another aspect the present invention relates to a hydrolysable polymer according wherein R₄ is selected from H and methyl.

In another aspect the present invention relates to a hydrolysable polymer according to wherein R₄ is H.

In another aspect the present invention relates to a hydrolysable polymer comprising a hindered bond functional group corresponding to the following formula (II)

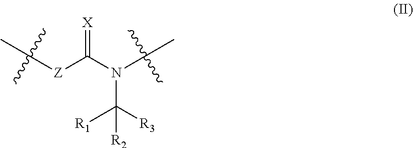

wherein X is O or S; Z is O, S, or NR₄; and R₁, R₂, R₃, and R₄ are independently selected from the group consisting of (C₁-C₂₀)alkyl, (C₄-C₁₀)cyclolalkyl, (C₁-C₂₀)alkyl(C₄-C₁₀)cycloalkyl, (C₁-C₂₀)alkyl(C₄-C₁₀)cycloalkyl(C₁-C₂₀)alkyl, (C₁-C₂₀)alkyl(C₆-C₁₀)aryl(C₁-C₂₀)alkyl, (C₂-C₂₀)alkyl-PEG-(C₂-C₂₀)alkyl, and H, and combinations thereof.

In another aspect the present invention relates to a hydrolysable polymer wherein X is O.

In another aspect the present invention relates to a hydrolysable polymer wherein R₁, R₂, R₃, are each methyl.

In another aspect the present invention relates to a hydrolysable polymer wherein Z is NR₄.

In another aspect the present invention relates to a hydrolysable polymer wherein R₄ is selected from H and methyl.

In another aspect the present invention relates to a hydrolysable polymer wherein R₄ is H.

In another aspect the present invention relates to a hydrolysable polymer comprising a hindered urea bond functional group corresponding to the following formula (III)

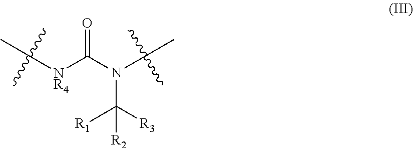

wherein R₁, R₂, R₃, and R₄ are independently selected from the group consisting of (C₁-C₂₀)alkyl, (C₄-C₁₀)cyclolalkyl, (C₁-C₂₀)alkyl(C₄-C₁₀)cycloalkyl, (C₁-C₂₀)alkyl(C₄-C₁₀)cycloalkyl(C₁-C₂₀)alkyl, (C₁-C₂₀)alkyl(C₆-C₁₀)aryl(C₁-C₂₀)alkyl, (C₂-C₂₀)alkyl-PEG-(C₂-C₂₀)alkyl, and H, and combinations thereof.

In another aspect the present invention relates to a hydrolysable polymer wherein R₁, R₂, and R₃ are each methyl.

In another aspect the present invention relates to a hydrolysable polymer wherein R₄ is H.

In another aspect the present invention relates to a hydrolysable polymer wherein the hindered bond or the hindered urea bond functional group has a K_{eq} less than 1×10⁶ M⁻¹ and a k₋₁ greater than 0.1 h⁻¹.

In another aspect the present invention relates to a hydrolysable polymer wherein the polymer exhibits at least 10% bond hydrolysis at 24 hours at 37° C.

In another aspect the present invention relates to a hydrolysable polymer wherein the polymer exhibits complete dissolution in an aqueous medium within 10 days.

In another aspect the present invention relates to a hydrolysable polymer wherein the dissolution occurs at normal room temperature.

In another aspect the present invention relates to a biodegradable packaging material comprising a hydrolysable polymer.

In another aspect the present invention relates to a drug delivery system comprising a hydrolysable polymer.

In another aspect the present invention relates to a medical device comprising a hydrolysable polymer.

In another aspect the present invention relates to a medical device wherein the medical device is an implantable medical device.

In another aspect the present invention relates to a surgical suture comprising a hydrolysable polymer.

In another aspect the present invention relates to a scaffold for tissue regeneration comprising a hydrolysable polymer.

In another aspect the present invention relates to a process for making a hydrolysable polymer comprising a hindered bond functional group, wherein the hindered bond functional group corresponds to the following formula (I)

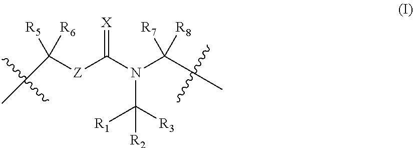

wherein X is O or S; Z is O, S, or NR₄; and R₁, R₂, R₃, and R₄ are independently selected from the group consisting of (C₁-C₂₀)alkyl, (C₄-C₁₀)cyclolalkyl, (C₁-C₂₀)alkyl(C₄-C₁₀)cycloalkyl, (C₁-C₂₀)alkyl(C₄-C₁₀)cycloalkyl(C₁-C₂₀)alkyl, (C₁-C₂₀)alkyl(C₆-C₁₀)aryl(C₁-C₂₀)alkyl, (C₂-C₂₀)alkyl-PEG-(C₂-C₂₀)alkyl, and H, and combinations thereof.

In another aspect the present invention relates to a process for making a hydrolysable polymer comprising a hindered bond functional group, wherein the hindered bond functional group corresponds to the following formula (II)

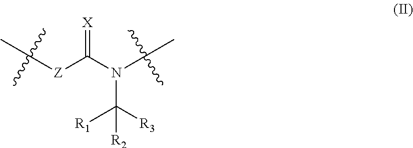

wherein X is O or S; Z is O, S, or NR₄; and R₁, R₂, R₃, and R₄ are independently selected from the group consisting of (C₁-C₂₀)alkyl, (C₄-C₁₀)cyclolalkyl, (C₁-C₂₀)alkyl(C₄-C₁₀)cycloalkyl, (C₁-C₂₀)alkyl(C₄-C₁₀)cycloalkyl(C₁-C₂₀)alkyl, (C₁-C₂₀)alkyl(C₆-C₁₀)aryl(C₁-C₂₀)alkyl, (C₂-C₂₀)alkyl-PEG-(C₂-C₂₀)alkyl, and H, and combinations thereof.

In another aspect the present invention relates to a polymer of the formula (IV)

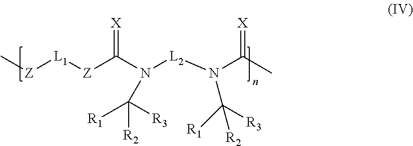

wherein each X is independently selected from O or S; each Z is independently selected from O, S, or NR₄; each R₁, R₂, R₃, and R₄ are independently selected from the group consisting of (C₁-C₂₀)alkyl, (C₄-C₁₀)cyclolalkyl, (C₁-C₂₀)alkyl(C₄-C₁₀)cycloalkyl, (C₁-C₂₀)alkyl(C₄-C₁₀)cycloalkyl(C₁-C₂₀)alkyl, (C₁-C₂₀)alkyl(C₆-C₁₀)aryl(C₁-C₂₀)alkyl, (C₂-C₂₀)alkyl-PEG-(C₂-C₂₀)alkyl, and H, and combinations thereof, and combinations thereof; L₁ and L₂ are independently selected from a linear, branched or network polymer or a small molecule linker, (C₂-C₂₀)alkyl, (C₄-C₁₀)cycloalkyl, (C₁-C₂₀)alkyl(C₄-C₁₀)cycloalkyl, (C₁-C₂₀)alkyl(C₄-C₁₀)cycloalkyl(C₁-C₂₀)alkyl, (C₁-C₂₀)alkyl(C₆-C₁₀)aryl(C₁-C20)alkyl, and (C₂-C₂₀)alkyl-PEG-(C₂-C₂₀)alkyl, and combinations thereof; and n is about 5 to about 500.

In another aspect the present invention relates to a polymer of formula (IV) wherein X is O.

In another aspect the present invention relates to a polymer of formula (IV) wherein R₁, R₂, R₃, are each methyl.

In another aspect the present invention relates to a polymer of formula (IV) wherein Z is NR₄.

In another aspect the present invention relates to a polymer of formula (IV) wherein R₄ is selected from H and methyl.

In another aspect the present invention relates to a polymer of formula (IV) wherein R4 is H.

In another aspect the present invention relates to a polymer of the formula (V)

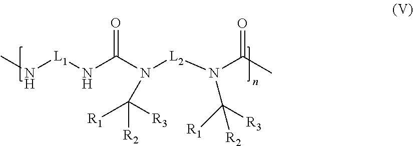

wherein each R₁, R₂, R₃, are independently selected from the group consisting of (C₁-C₂₀)alkyl, (C₄-C₁₀)cyclolalkyl, (C₁-C₂₀)alkyl(C₄-C₁₀)cycloalkyl, (C₁-C₂₀)alkyl(C₄-C₁₀)cycloalkyl(C₁-C₂₀)alkyl, (C₁-C₂₀)alkyl(C₆-C₁₀)aryl(C₁-C₂₀)alkyl, (C₂-C₂₀)alkyl-PEG-(C₂-C₂₀)alkyl, and H, and combinations thereof; L₁ and L₂ are independently selected from a linear, branched or network polymer or a small molecule linker, (C₂-C₂₀)alkyl, (C₄-C₁₀)cyclolalkyl, (C₁-C₂₀)alkyl(C₄-C₁₀)cycloalkyl, (C₁-C₂₀)alkyl(C₄-C₁₀)cycloalkyl(C₁-C₂₀)alkyl, (C₁-C₂₀)alkyl(C₆-C₁₀)aryl(C₁-C₂₀)alkyl, (C₂-C₂₀)alkyl-PEG-(C₂-C₂₀)alkyl, and combinations thereof; and n is about 5 to about 500.

In another aspect the present invention relates to a polymer of formula (V) wherein R₁, R₂, R₃, are each methyl.

In another aspect the present invention relates to a method for preparing a polymer containing a hindered amine functional group, comprising the steps of: (a) reacting a polymer containing a free hydroxyl or primary amino group with a divinyl sulfone to give an ether or amino substituted vinyl sulfone containing polymer; and (b) reacting the resultant ether or amino substituted vinyl sulfone containing polymer with a hindered primary amino compound to give the polymer containing the hindered amine functional group.

In another aspect the present invention relates to a method further comprising the step of, (c) reacting the resultant polymer containing the hindered amine functional group with an isocyanate crosslinking agent.

In another aspect the present invention relates to a method for preparing a polymer containing a hindered amine functional group, comprising the step of: (a) reacting a polymer containing an allyl or benzylic functional group with a hindered primary amino compound to give the polymer containing the hindered amine functional group.

In another aspect the present invention relates to a method for preparing a polymer containing a hindered amine functional group, comprising the step of: reacting a polymer containing the following functional group (A)

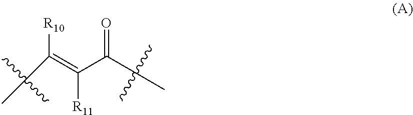

wherein R₁₀ and R₁₁ are independently selected from H or C₁-C₆ linear, branched or cyclic alkyl, with a hindered primary amino compound to give the polymer containing the hindered amine functional group.

In another aspect the present invention relates to a method for preparing a polymer containing a hindered amine functional group, comprising the step of: reacting a polymer containing an allylic or benzylic functional group with a hindered primary amino compound to give the polymer containing the hindered amine functional group, wherein the hindered amine functional group is located at the allylic or benzylic position of the allylic or benzylic functional group.

In another aspect the present invention relates to a method for preparing a polymer containing a hindered amine functional group, comprising the step of: reacting a polymer containing a primary amino group with a bulky or hindered alkylating agent to give the polymer containing the hindered amine functional group.

In another aspect the present invention relates to a method for preparing a polymer containing a hindered amine functional group, comprising the steps of: (a) reacting a polymer containing a primary amino group with a ketone or an aldehyde to give an imine substituted polymer; and (b) reducing the imine substituted polymer to give the polymer containing the hindered amine functional group.

### Definitions

As used herein, the following terms have the indicated meanings unless expressly stated to the contrary:

The term “bulky” as used herein refers to a group or substituent having steric hindrance, especially where the bulky group provides dynamic exchange within a polymer, as described herein. The term “bulky” may be applied to an alkyl, aryl, amino, or other group. Exemplary “bulky alkyl” groups include, but are not limited to isopropyl, tert-butyl, neopentyl, and adamantly. Exemplary “bulky aryl” groups include, but are not limited to, trityl, biphenyl, naphthayl, indenyl, anthracyl, fluorenyl, azulenyl, phenanthrenyl, and pyrenyl. Exemplary “bulky amine” groups include, but are not limited to, tertiary amines substituted with one or more bulky akyl or bulky aryl groups, such as two tert-butyl groups. Exemplary “bulky amide” groups include, but are not limited to, carboxyl groups coupled to a bulky amine.

The term “dynamic bond” or “dynamic bond functional group” refers to a bond or chemical group or functional group that can reversibly form and dissociate. The term “dynamic urea bond” as used herein refers to a urea bond in the polymers herein that can reversibly form and dissociate. Ureas can be represented by the following chemical structure (i):

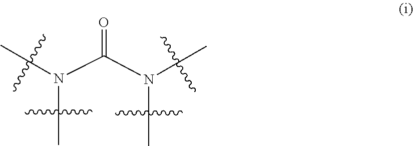 [SMILES: *N(*)C(=O)N(*)*]

It should be recognized that ureas represent a subset of other oxygen, nitrogen, and sulfur-containing variants, as represented by the more general formula (ii), which are also considered as part of the present invention:

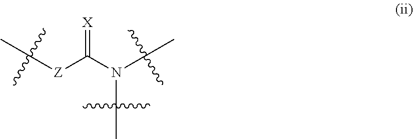

wherein X is O or S; Z is O, S, or NR4, wherein R₄ is selected from the group consisting of (C₁-C₂₀)alkyl, (C₄-C₁₀)cyclolalkyl, (C₁-C₂₀)alkyl(C₄-C₁₀)cycloalkyl, (C₁-C₂₀)alkyl(C₄-C₁₀)cycloalkyl(C₁-C₂₀)alkyl, C₂-C₂₀)alkyl-PEG-(C₂-C₂₀)alkyl, and H, and combinations thereof.

The hindered urea bonds and the polymers of the present invention are such that the) r nitrogen atoms of the urea moiety, e.g., as depicted in formula (i) or the more general moieties, e.g., as depicted in formula (ii), are not directly bonded to an aromatic moiety. In other words, for the urea moiety or for the more general moieties, the nitrogen atom or atoms attached to the carbonyl (or carbonyl equivalent of the more general moieties) are not also directly attached to an aromatic moiety.

The term “highly crosslinked” as used herein refers to a polymer that is extensively cross linked. In such polymers the average linker length between each crosslinking point ranges from 1 to about 100 atoms.

The term “hindered” as used herein refers to a chemical group, such as a hindered bond functional group. In the present invention, a hindered bond functional group includes urea bonds of the present invention that are sterically hindered by one or more bulky groups or substitutents. Furthermore, it is recognized that additional substituents can be described to flank these bonds as further shown in formula (I).

The term “hindered urea bond” as used herein refers to a urea bond in a polymer of the present invention that is hindered with one or more bulky groups. It is recognized the “hindered urea bonds” represent a subset of various oxygen, sulfur, and nitrogen-substituted ureas that are considered part of the present invention.

The term “hydrolysable” as used herein means that the hindered bonds or functional groups, such as the hindered urea bonds, can be broken down, or undergo hydrolysis in the presence of water. In its common usage, hydrolysis means the cleavage of chemical bonds by the addition of water. In the presence invention, the hindered bond can undergo hydrolysis.

The term “reversible polymer” as used herein refers to a polymer with blocks or repeating units containing non-covalent or dynamic covalent bonds that can reversibly form and dissociate.

The term “self-healing” as used herein refers to the property of a reversible polymer that autonomously repairs damage caused by mechanical usage over time and recovers substantially its original modulus and strength.

The term “shape memory polymer” as used herein refers to a polymeric smart material that has the ability to return from a deformed state, i.e. its temporary shape, to its original or permanent shape, induced by a stimulus or trigger.

The term “acyl,” as used herein alone or as part of another group, denotes the moiety formed by removal of the hydroxy group from the group COOH of an organic carboxylic acid, e.g., RC(O)—, wherein R is R1, R1O—, R1R2N—, or R1S—, R1 is hydrocarbyl, heterosubstituted hydrocarbyl, or heterocyclo, and R2 is hydrogen, hydrocarbyl, or substituted hydrocarbyl.

The term “acyloxy,” as used herein alone or as part of another group, denotes an acyl group as described above bonded through an oxygen linkage (O), e.g., RC(O)O— wherein R is as defined in connection with the term “acyl.”

The term “alkyl” refers to a branched or unbranched hydrocarbon having, for example, from 1-20 carbon atoms, and often 1-12, 1-10, 1-8, 1-6, or 1-4 carbon atoms. Examples include, but are not limited to, methyl, ethyl, 1-propyl, 2-propyl (iso-propyl), 1-butyl, 2-methyl-1-propyl (isobutyl), 2-butyl (sec-butyl), 2-methyl-2-propyl (t-butyl), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, hexyl, octyl, decyl, dodecyl, and the like. The alkyl can be unsubstituted or substituted, for example, with a substituent described below. The alkyl can also be optionally partially or fully unsaturated. As such, the recitation of an alkyl group includes both alkenyl and alkynyl groups. The alkyl can be a monovalent hydrocarbon radical, as described and exemplified above, or it can be a divalent hydrocarbon radical (i.e., an alkylene). In some embodiments, “alkyl” refers to a fully saturated alkyl. In other embodiments, “alkyl” is branched or unbranched, and is non-cyclic.

The term “alkenyl” as used herein describes groups which are preferably lower alkenyl containing from two to eight carbon atoms in the principal chain and up to 20 carbon atoms. They may be straight or branched chain and include ethenyl, propenyl, isopropenyl, butenyl, isobutenyl, hexenyl, and the like.

The term “alkynyl” as used herein describes groups which are preferably lower alkynyl containing from two to eight carbon atoms in the principal chain and up to 20 carbon atoms. They may be straight or branched chain and include ethynyl, propynyl, butynyl, isobutynyl, hexynyl, and the like.

The term “aliphatic” as used herein refers to a chemical compound belonging to the organic class in which the atoms are not linked together to form an aromatic ring. One of the major structural groups of organic molecules, the aliphatic compounds include the alkanes, alkenes, and alkynes, including linear, branched, and cyclic variants, and substances derived from them—actually or in principle—by replacing one or more hydrogen atoms by atoms of other elements or groups of atoms.

The term “aromatic” as used herein alone or as part of another group denotes optionally substituted homo- or heterocyclic conjugated planar ring or ring system comprising delocalized electrons. These aromatic groups are preferably monocyclic (e.g., furan or benzene), bicyclic, or tricyclic groups containing from 5 to 14 atoms in the ring portion. The term “aromatic” encompasses “aryl” groups defined below.

The term “aryl” refers to an aromatic hydrocarbon group derived from the removal of at least one hydrogen atom from a single carbon atom of a parent aromatic ring system. The radical attachment site can be at a saturated or unsaturated carbon atom of the parent ring system. The aryl group can have from 6 to 30 carbon atoms, for example, about 6-10 carbon atoms. The aryl group can have a single ring (e.g., phenyl) or multiple condensed (fused) rings, wherein at least one ring is aromatic (e.g., naphthyl, dihydrophenanthrenyl, fluorenyl, or anthryl). Typical aryl groups include, but are not limited to, radicals derived from benzene, naphthalene, anthracene, biphenyl, and the like. The aryl can be unsubstituted or optionally substituted, as described for alkyl groups.

The terms “carbocyclo” or “carbocyclic” as used herein alone or as part of another group denote optionally substituted, aromatic or non-aromatic, homocyclic ring or ring system in which all of the atoms in the ring are carbon, with preferably 5 or 6 carbon atoms in each ring. Exemplary substituents include one or more of the following groups: hydrocarbyl, substituted hydrocarbyl, alkyl, alkoxy, acyl, acyloxy, alkenyl, alkenoxy, aryl, aryloxy, amino, amido, acetal, carbamyl, carbocyclo, cyano, ester, ether, halogen, heterocyclo, hydroxy, keto, ketal, phospho, nitro, and thio.

The term “cycloalkyl” refers to cyclic alkyl groups of, for example, from 3 to 10 carbon atoms having a single cyclic ring or multiple condensed rings. Cycloalkyl groups include, by way of example, single ring structures such as cyclopropyl, cyclobutyl, cyclopentyl, cyclooctyl, and the like, or multiple ring structures such as adamantyl, and the like. The cycloalkyl can be unsubstituted or substituted. The cycloalkyl group can be monovalent or divalent, and can be optionally substituted as described for alkyl groups. The cycloalkyl group can optionally include one or more cites of unsaturation, for example, the cycloalkyl group can include one or more carbon-carbon double bonds, such as, for example, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, and the like.

The term “heteroatom” refers to atoms other than carbon and hydrogen.

The term “heteroaromatic” as used herein alone or as part of another group denotes optionally substituted aromatic groups having at least one heteroatom in at least one ring, and preferably 5 or 6 atoms in each ring. The heteroaromatic group preferably has 1 or 2 oxygen atoms and/or 1 to 4 nitrogen atoms in the ring, and is bonded to the remainder of the molecule through a carbon. Exemplary groups include furyl, benzofuryl, oxazolyl, isoxazolyl, oxadiazolyl, benzoxazolyl, benzoxadiazolyl, pyrrolyl, pyrazolyl, imidazolyl, triazolyl, tetrazolyl, pyridyl, pyrimidyl, pyrazinyl, pyridazinyl, indolyl, isoindolyl, indolizinyl, benzimidazolyl, indazolyl, benzotriazolyl, tetrazolopyridazinyl, carbazolyl, purinyl, quinolinyl, isoquinolinyl, imidazopyridyl, and the like. Exemplary substituents include one or more of the following groups: hydrocarbyl, substituted hydrocarbyl, alkyl, alkoxy, acyl, acyloxy, alkenyl, alkenoxy, aryl, aryloxy, amino, amido, acetal, carbamyl, carbocyclo, cyano, ester, ether, halogen, heterocyclo, hydroxy, keto, ketal, phospho, nitro, and thio.

The terms “heterocyclo” or “heterocyclic” as used herein alone or as part of another group denote optionally substituted, fully saturated or unsaturated, monocyclic or bicyclic, aromatic or non-aromatic groups having at least one heteroatom in at least one ring, and preferably 5 or 6 atoms in each ring. The heterocyclo group preferably has 1 or 2 oxygen atoms and/or 1 to 4 nitrogen atoms in the ring, and is bonded to the remainder of the molecule through a carbon or heteroatom. Exemplary heterocyclo groups include heteroaromatics as described above. Exemplary substituents include one or more of the following groups: hydrocarbyl, substituted hydrocarbyl, alkyl, alkoxy, acyl, acyloxy, alkenyl, alkenoxy, aryl, aryloxy, amino, amido, acetal, carbamyl, carbocyclo, cyano, ester, ether, halogen, heterocyclo, hydroxy, keto, ketal, phospho, nitro, and thio.

The terms “hydrocarbon” and “hydrocarbyl” as used herein describe organic compounds or radicals consisting exclusively of the elements carbon and hydrogen. These moieties include alkyl, alkenyl, alkynyl, and aryl moieties. These moieties also include alkyl, alkenyl, alkynyl, and aryl moieties optionally substituted with other aliphatic or cyclic hydrocarbon groups, such as alkaryl, alkenaryl and alkynaryl. Unless otherwise indicated, these moieties preferably comprise 1 to 20 carbon atoms.

The “substituted hydrocarbyl” moieties described herein are hydrocarbyl moieties which are substituted with at least one atom other than carbon, including moieties in which a carbon chain atom is substituted with a heteroatom such as nitrogen, oxygen, silicon, phosphorous, boron, or a halogen atom, and moieties in which the carbon chain comprises additional substituents. These substituents include alkyl, alkoxy, acyl, acyloxy, alkenyl, alkenoxy, aryl, aryloxy, amino, amido, acetal, carbamyl, carbocyclo, cyano, ester, ether, halogen, heterocyclo, hydroxy, keto, ketal, phospho, nitro, and thio.

Generally, the term “substituted” indicates that one or more hydrogen atoms on the group indicated in the expression using “substituted” is replaced with a “substituent”. The number referred to by ‘one or more’ can be apparent from the moiety one which the substituents reside. For example, one or more can refer to, e.g., 1, 2, 3, 4, 5, or 6; in some embodiments 1, 2, or 3; and in other embodiments 1 or 2. The substituent can be one of a selection of indicated groups, or it can be a suitable group known to those of skill in the art, provided that the substituted atom's normal valency is not exceeded, and that the substitution results in a stable compound. Suitable substituent groups include, e.g., alkyl, alkenyl, alkynyl, alkoxy, halo, haloalkyl, hydroxy, hydroxyalkyl, aryl, aroyl, (aryl)alkyl (e.g., benzyl or phenylethyl), heteroaryl, heterocycle, cycloalkyl, alkanoyl, alkoxycarbonyl, amino, alkylamino, dialkylamino, trifluoromethyl, trifluoromethoxy, trifluoromethylthio, difluoromethyl, acylamino, nitro, carboxy, carboxyalkyl, keto, thioxo, alkylthio, alkylsulfinyl, alkylsulfonyl, arylsulfinyl, arylsulfonyl, heteroarylsulfinyl, heteroarylsulfonyl, heterocyclesulfinyl, heterocyclesulfonyl, phosphate, sulfate, hydroxyl amine, hydroxyl (alkyl)amine, and cyano. Additionally, suitable substituent groups can be, e.g., —X, —R, —O—, —OR, —SR, —S—, —NR₂, —NR3, ═NR, —CX3, —CN, —OCN, —SCN, —N═C═O, —NCS, —NO, —NO2, ═N2, —N3, —NC(═O)R, —C(=O)R, —C(═O)NRR, —S(═O)2O—, —S(50 O)2H, —S(═O)2R, —OS(═O)2R, —S(═O)2NR, —S(═O)R, —OP(═O)(OR)2, —P(═O)(OR)2, —OP(═O)(OH)(OR), —P(═O)(OH)(OR), —P(═O)(O—)2, —P(═O)(OH)2, —(═O)R, —C(═O)X, —C(S)R, —C(O)OR, —C(O)O—, —C(S)OR, —C(O)SR, —C(S)SR, —C(O)NRR, —C(S)NRR, or —C(NR)NRR, where each X is independently a halogen (“halo”): F, Cl, Br, or I; and each R is independently H, alkyl, aryl, (aryl)alkyl (e.g., benzyl), heteroaryl, (heteroaryl)alkyl, heterocycle, heterocycle(alkyl), or a protecting group. As would be readily understood by one skilled in the art, when a substituent is keto (═O) or thioxo (═S), or the like, then two hydrogen atoms on the substituted atom are replaced. In some embodiments, one or more of the substituents above are excluded from the group of potential values for sub stituents on the substituted group.

The term “interrupted” indicates that another group is inserted between two adjacent carbon atoms (and the hydrogen atoms to which they are attached (e.g., methyl (CH3), methylene (CH2) or methine (CH))) of a particular carbon chain being referred to in the expression using the term “interrupted, provided that each of the indicated atom's normal valency is not exceeded, and that the interruption results in a stable compound. Suitable groups that can interrupt a carbon chain include, e.g., with one or more non-peroxide oxy (—O—), thio (—S—), imino (—N(H)—), methylene dioxy (—OCH2O—), carbonyl (—C(═O)—), carboxy (—C(═O)O—), carbonyldioxy (—OC(═O)O—), carboxylato (—OC(═O)—), imine (C═NH), sulfinyl (SO) and sulfonyl (SO2). Alkyl groups can be interrupted by one or more (e.g., 1, 2, 3, 4, 5, or about 6) of the aforementioned suitable groups. The site of interruption can also be between a carbon atom of an alkyl group and a carbon atom to which the alkyl group is attached. An alkyl group that is interrupted by a heteroatom therefor forms a heteroalkyl group.

Substituents can include cycloalkylalkyl groups. “Cycloalkylalkyl” may be defined as a cycloalkyl-alkyl-group in which the cycloalkyl and alkyl moieties are as previously described. Exemplary monocycloalkylalkyl groups include cyclopropylmethyl, cyclopentylmethyl, cyclohexylmethyl and cycloheptylmethyl.

It is intended that the groups such as “M”, “X” [other than when “X” is ═X as in formulas (I), (II), and (IV) and chemical structure (ii)], “L”, “L₁”, and “L₂” are selected from a single bond, (C₁-C₂₀)alkyl, (C₄-C₁₀)cyclolalkyl, (C₁-C₂₀)alkyl(C₄-C₁₀)cycloalkyl, (C₁-C₂₀)alkyl(C₄-C₁₀)cycloalkyl(C₁-C₂₀)alkyl, (C₁-C₂₀)alkyl(C₆-C₁₀)aryl(C₁-C₂₀)alkyl, and (C₂-C₂₀)alkyl-PEG-(C₂-C₂₀)alkyl, and the like, and are written as such for simplicity but are intended to be “difunctional” radicals or moieties that are connected at each end and in either orientation, unless indicated specifically otherwise. For example, (C₁-C₂₀)alkyl is intended to mean the difunctional radical —(C₁-C₂₀)alkyl -, an example of which is —(CH₂)₅. These difunctional radicals are distinguished from the monofunctional radicals such as R₁, R₂, R₃, R₄, R₅, R₆, R₇, R₈, R₁₀, and R₁₁, which are only connected at one end.

### Dynamic Bonds of the Polymers and Precursors

The polymers of the present invention comprise dynamic bonds such as hindered urea bonds. Furthermore, the precursors used to make these polymers can in some instances comprise these dynamic bonds or chemical groups that are used to form these dynamic bonds.

For example, the polymers comprise a hindered bond functional group corresponding to the following formula (I)

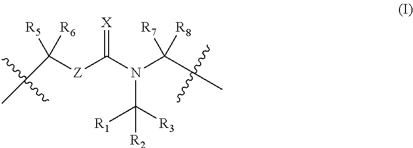

wherein X is O or S; Z is O, S, or NR₄; and R₁, R₂, R₃, R₄, R₅, R₆, R₇, and R₈ are independently selected from the group consisting of (C₁-C₂₀)alkyl, (C₄-C₁₀)cyclolalkyl, (C₁-C₂₀)alkyl(C₄-C₁₀)cycloalkyl, (C₁-C₂₀)alkyl(C₄-C₁₀)cycloalkyl(C₁-C₂₀)alkyl, (C₂-C₂₀)alkyl-PEG-(C₂-C₂₀)alkyl, and H.

Alternatively, the polymers comprise a hindered bond functional group corresponding to the following formula (II)

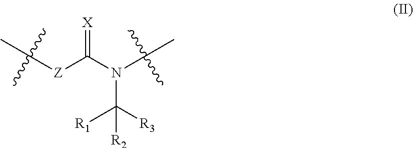

wherein X is O or S; Z is O, S, or NR₄; and R₁, R₂, R₃, and R₄ are independently selected from the group consisting of (C₁-C₂₀)alkyl, (C₄-C₁₀)cyclolalkyl, (C₁-C₂₀)alkyl(C₄-C₁₀)cycloalkyl, (C₁-C₂₀)alkyl(C₄-C₁₀)cycloalkyl(C₁-C₂₀)alkyl, (C₂-C₂₀)alkyl-PEG-(C₂-C₂₀)alkyl, and H.

Alternatively, the polymers comprise a hindered urea bond functional group corresponding to the following formula (III)

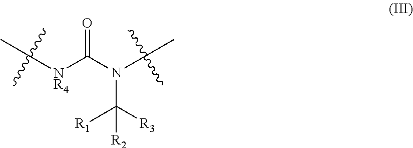

wherein R₁, R₂, R₃, and R₄ are independently selected from the group consisting of (C₁-C₂₀)alkyl, (C₄-C₁₀)cyclolalkyl, (C₁-C₂₀)alkyl(C₄-C₁₀)cycloalkyl, (C₁-C₂₀)alkyl(C₄-C₁₀)cycloalkyl(C₁-C₂₀)alkyl, (C₂-C₂₀)alkyl-PEG-(C₂-C₂₀)alkyl, and H.

### Polymers

The polymers of the present invention comprise dynamic bonds such as dynamic urea bonds, and more particularly “hindered urea bonds” or “HUBs”. The present invention provides polymers having dynamic urea bonds. These polymers include: (a) malleable, repairable, and reprogrammable shape memory polymers having HUBs and (b) reversible or degradable (e.g., via hydrolysis or aminolysis) linear, branched or network polymers having HUBs. With respect to the malleable, repairable, and reprogrammable shape memory polymers these include polymers containing other polymer generating functionality that now incorporate these HUB s, as well as to highly crosslinked polymers, and to polymers that are readily reprogrammed. For example, the degradation kinetics could be directly controlled by substituents bulkiness. In contrast to traditional hydrolysable polymers, the HUB containing polymers of the present invention could be synthesized form monomers by simple mixing without catalysts. Further background on earlier examples of polymers with dynamic urea bonds is disclosed in PCT Publication WO 2014/144539 A2, to The Board of Trustees of the University of Illinois, published Sep. 18, 2014, which is incorporated by reference herein in its entirety.

### Malleable, Recyclable, and Healable Thermoset Polymers

Highly cross-linked thermoset polymers, which offer robust mechanical properties and solvent resistance, have been studied as matrices for composites, foamed structures, structural adhesives, insulators for electronic packaging, etc. However, highly covalent cross-linked network polymers generally lack the ability to be recycled, processed and self-healed after unwanted cracks have generated. Compared to low cross-linked density polymers, such as poly(urea-urethanes) (PUUs), the highly cross-linked polymers would have different properties. For example, low cross-linked density polymers are difficult to use as structural materials in many area because of their low Yang's modulus (˜1MPa). Here we have developed a new class of stiff and strong transparent poly(urea-urethane) (tert-butylamino)ethanol thermoset (PUU-TBAE) polymers based on dynamic covalent hindered urea bonds with high Young's modulus RE), ˜3.5 GPa by nanoindendation, 1.9 GPa by dynamic mechanical analysis (DMA)], high hardness (˜250 MPa) and high breaking strength (˜39.5 MPa). These PUU-TBAE thermoset polymers have an excellent malleability which essentially behaves like a classic thermoset under ambient conditions, yet can be reprocessed by application of heat. Furthermore PUU-TBAE thermoset polymers have self-healing properties under mild or ambient conditions, and recyclability, which can be recovered from a mixture of traditional thermoplastics and thermosets. These properties mean that environmentally compatible (“green”), low temperature processing conditions can be used for this important class of cross-linked functional polymers.

### Malleable and Reprogrammable Shape Memory Polymers (SMPs)

Shape-memory polymers (SMPs) are polymeric smart materials that have the ability to return from a deformed state (temporary shape) to their original (permanent) shape induced by an external stimulus (trigger), such as temperature change. Traditionally, the structures of SMP are covalently cross-linked polymers with switching segment that has the ability to soften past a certain transition temperature. The covalent cross-linking fixed the permanent shape, while the switching segment is responsible for the temporary shape. However, once the permanent shape was set by covalent cross-linking, materials can no longer be reprogrammed or processed. Herein, we incorporated HUB, a type of dynamic urea bond, as the covalent cross-linker in the design of SMP (HUB -SMP). The dynamic exchange of HUB is slow enough under the triggered-shape-changing conditions to retain the ‘permanent’ shape. But under higher temperature or longer incubation time, the ‘permanent’ shape could be reprogrammed due to the dynamic exchange of HUB cross-linker. Also the dynamic property of HUB facilitates the processing of SMP with heat extrusion method, which makes it potential as a type of ‘4D printing’ (‘3D printable’ shape memory) materials.

Traditional SMPs cannot be processed, reprogrammed or recycled after the permanent shape is set by covalent cross-linking. Our goal is to incorporate HUBs as dynamic cross-linker for the design of malleable and shape reprogrammable SMP.

The described designs are SMPs with new type of dynamic covalent cross-linker HUBs. The new composition improves the existing SMPs by giving them malleable and reprogrammable properties.

Traditional SMPs cannot be processed or reprogrammed after the permanent shape is set by covalent cross-linking. Our new design solves the issue by giving them malleable and shape reprogrammable properties through the incorporation of HUB s capable of dynamic exchange. The new materials can be reprogrammed to any shape even after curing step. They could be molded through hot press or heat extrusion methods. And they could be easily recycled after use. Furthermore, similar to highly developed polyurethane/polyurea industry, synthesis of HUBs based SMPs is very straightforward through simple mixing of isocyanate and hindered amine precursors.

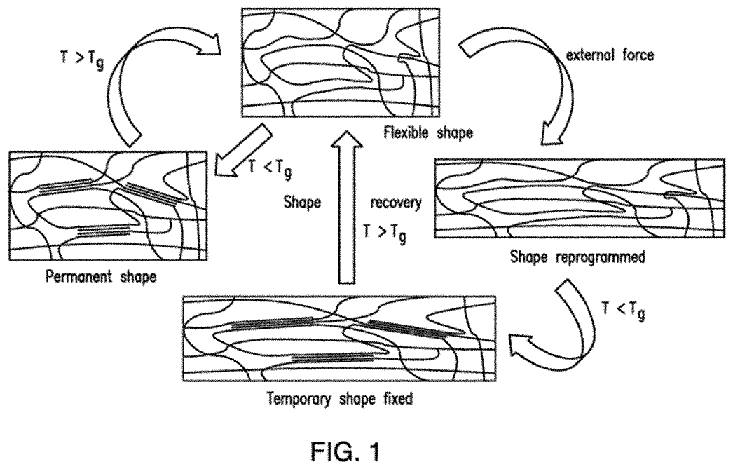

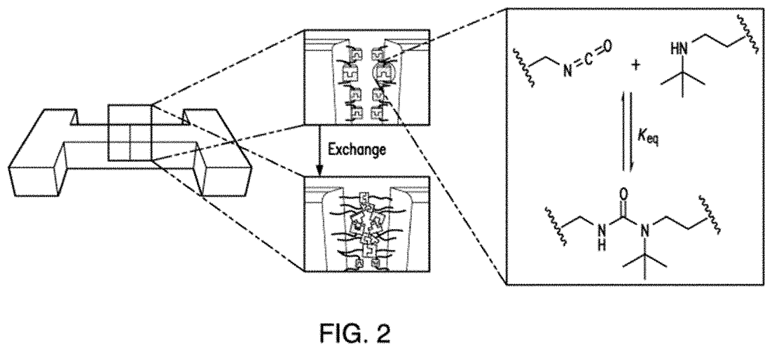

Shape-memory polymers (SMPs) are polymeric smart materials that have the ability to return from a deformed state (temporary shape) to their original (permanent) shape induced by an external stimulus (trigger), such as temperature change. Traditionally, the structures of SMP are covalently cross-linked polymers with switching segment that has the ability to soften past a certain transition temperature. The covalent cross-linking fixed the permanent shape, while the switching segment is responsible for the temporary shape. As shown in FIG. 1, after heating the sample above its transition temperature, the switching domain softens, and the material changes shape with applied force. If cooling down with the force kept, the switching domain gets fixed, which retains the sample shape even after applied force is removed. After that, if heating the sample above the transition temperature again, the switching domain softens, which leads to the recovery of sample's permanent shape.

However, once the permanent shape was set by covalent cross-linking, materials can no longer be reprogrammed or processed. Herein, we incorporated HUB, a type of dynamic urea bondl, as the covalent cross-linker in the design of SMP (HUB-SMP). The dynamic exchange of HUB is slow enough under the triggered-shape-changing conditions to retain the ‘permanent’ shape. But under higher temperature or longer incubation time, the ‘permanent’ shape could be reprogrammed due to the dynamic exchange of HUB cross-linker.

### Hydrolysable and Reversible Polymers

Hydrolysable polymers are widely used materials that have found numerous applications in the biomedical, agricultural, plastic, and packaging industries. The present invention provides hydrolysable polymers having dynamic bonds such as dynamic urea bonds.

The degradation kinetics could be directly controlled by substituent bulkiness. In contrast to traditional hydrolysable polymers, the HUB containing polymers of the present invention could be synthesized from monomers by simple mixing without catalysts.

Hydrolysable polymers are widely used materials that have found numerous applications in biomedical, agro-, plastic and packaging industrials. They usually contain ester and other hydrolysable bonds, such as anhydride, acetal, ketal or imine, in their backbone structures. Here, we report the first design of hydrolysable polyureas (HPUs) bearing dynamic hindered urea bonds (HUBs) that can reversibly dissociate to bulky amines and isocyanates, the latter of which can be further hydrolyzed by water, driving the equilibrium to facilitate the degradation of HPUs. HPU bearing 1-tert-butyl-1-ethylurea bonds that show high dynamicity (high bond dissociation rate), in the form of either linear polymers or cross-linked gels, can be completely degraded by water under mild conditions. Given the simplicity and low cost for the production of HPUs by simply mixing multifunctional bulky ureas and isocyanates, the versatility of their structures and the tunability of their degradation profiles, these materials are potentially of broad application.

Over the past few decades, hydrolysable polymeric materials have attracted numerous attentions in both academic and industrial settings. For example, the transient stability of hydrolysable polymers in aqueous solution is critical to their biomedical applications, such as in the design of drug delivery systems, scaffolds for tissue regeneration, surgical sutures, and transient medical devices and implants, which usually require short functioning time and complete degradation and clearance after use. They have also been applied in the design of controlled release systems in agroindustry, and degradable, environmentally friendly plastics and packaging materials. Polyesters are the most widely used, conventional hydrolysable materials. A large variety of other hydrolysable polymers bearing orthoester, acetal, ketal, aminal, hemiaminal, imine, phosphoester, and phosphazene bonds have also been reported. Syntheses of these polymers usually involve condensation polymerization of acyclic monomers or ring-opening polymerization of cyclic monomers, and these syntheses typically involve removal of byproducts, such as water, and use of high reaction temperature or metal catalysts, which can complicate preparation of the material.

Polyureas are commonly used as fiber, coating and adhesive materials. Polyureas can be readily synthesized via addition reaction of widely available, di- or multifunctional isocyanates and amines that do not require the use of catalysts and extreme reaction conditions and do not produce any byproducts. Urea is one of the most stable chemical bonds against further reactions including hydrolysis, due to the conjugation stabilization effects of its dual amide structure. However, urea bonds can be destabilized by incorporating bulky substituents to one of its nitrogen atoms, by means of disturbing the orbital co-planarity of the amide bonds that diminishes the conjugation effect. Urea bonds bearing a bulky substituent, or hindered urea bonds (HUBs), can reversibly dissociate into isocyanate and amines and show interesting dynamic property. The fast reversible reactions between HUBs and isocyanates/amines have been the basis in the design of self-healing polyureas. Because isocyanates can be subject to hydrolysis in aqueous solution to form amines and carbon dioxide, an irreversible process that shifts the equilibrium to favor the HUB dissociation reaction and eventually leads to irreversible and complete degradation of HUBs, can be used to design hydrolysable polymers. Herein, we report the development of HUB-based polyureas that can be hydrolyzed with hydrolytic degradation kinetics tunable by the steric hindrance of the HUB structures.

### Precursors

The present invention provides precursors for incorporation of HUB s into the polymers of the present invention. Examples of precursor monomers include the following.

Hindered amine precursors.

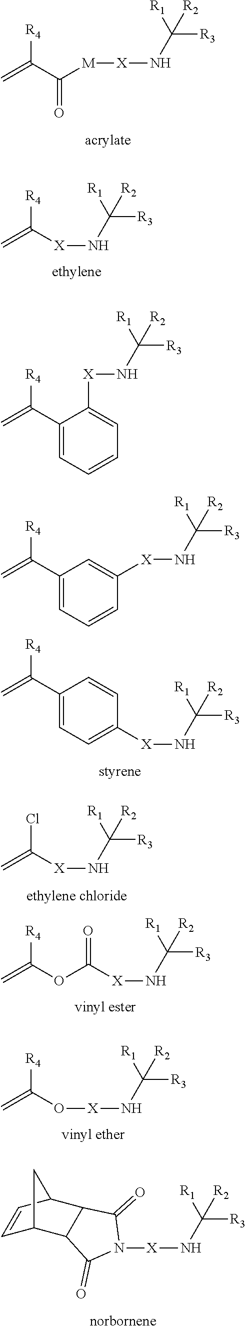

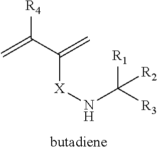

The hindered amine substituted monomers are such that the amino functional group is not directly attached to an aromatic group. In other words the hindered amine monomer is not an aromatic amine.

### Conversion of Other Polymers into HUB Containing Polymers

The polymers of the present invention can be made by converting other polymers, including readily available polymers into HUB containing polymers. For example, polymers with free hydroxyl or amino groups can be converted to HUB containing polymers. The following scheme illustrates such a process for a polymer containing amino groups. In this scheme: A depicts hyaluronic acid with side chains modified by sulfone groups. B depicts hyaluronic acid with side chains modified by hindered amine groups. C depicts hyaluronic acid with side chains modified by methacrylate groups containing hindered urea bonds between end groups and a polymeric backbone.

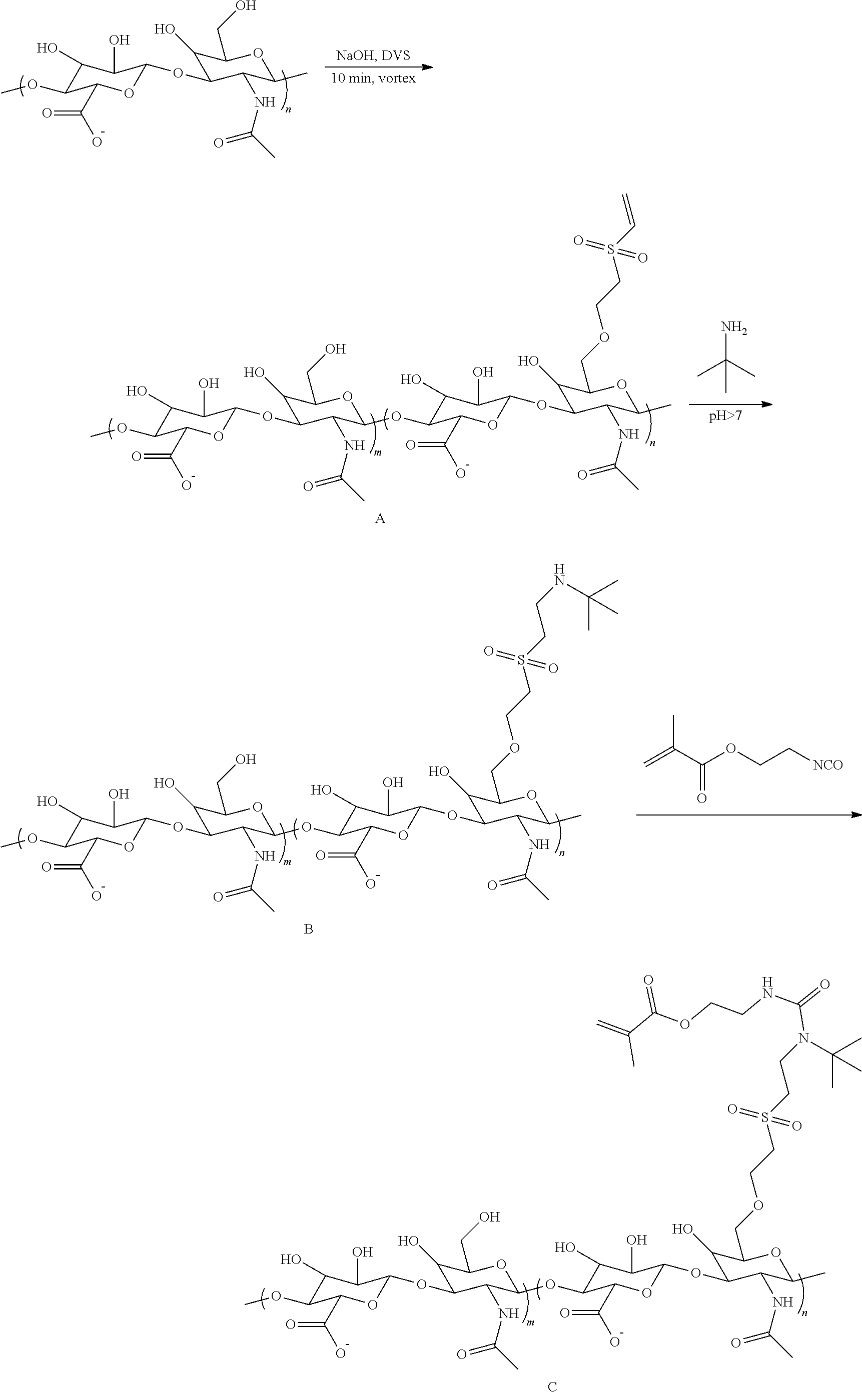

Alternatively, HUB containing polymers can be made by an addition process, such as a Michael addition to a polymer having unsaturated ester groups as illustrated by the following scheme to insert a hindered amine group. This hindered amine group can be further reacted with an isocyanate to yield a HUB.

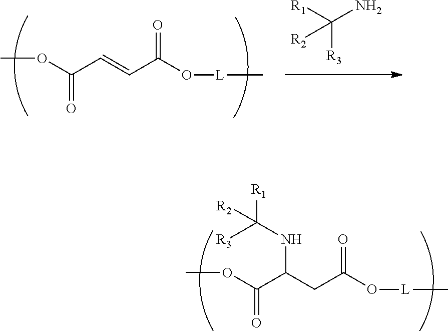

As another alternative, HUB containing polymers can be made by the radical amination of various polymeric materials. The hindered amine group can be further reacted with an isocyanate to yield a HUB.

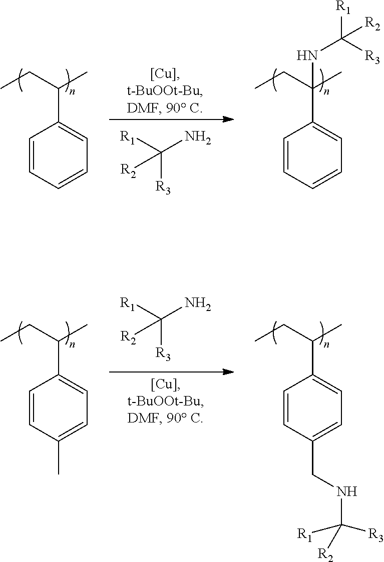

### Methods of Preparing Polymers

The disclosure further provides a method for preparing a copolymer comprising dynamic urea moieties. The method comprises contacting an alkyldiisocyanate and an alkyldiamine in solution, wherein the amines of the alkyldiamine comprise a tert-butyl substituent in a solvent system to form an oligourea. The oligourea is contacted with a trialkanolamine and a polyethylene glycol in the presence of a condensation reaction catalyst, thereby initiating cross-linking. The method provides a cross-linked poly(urea-urethane) polymer.

In one embodiment, the diisocyanate may be a C₂-C₁₂ diisocyanate. Exemplary diisocyantes include, but are not limited to, toluylene diisocyanate, diphenylmethane diisocyanate, dicyclohexylmethane diisocyanate, isophorone diisocyanate, trimethylhexane diisocyanate, cyclohexane diisocyanate, cyclohexanedimethylene diisocyanate, and tetramethylenexylylene diisocyanate. In some embodiments, the diisocyanate may be a C₂-C₁₂ diisocyanate.

Exemplary alkyldiamines include, but are not limited to, diprimary diamines, diamines containing one or two secondary amino groups with an alkyl substituent having from 1 to 8 carbon atoms attached to the N-atom, and a heterocyclic diamine. The diprimary aliphatic diamines may contain terminal amino groups. In some embodiments, the diamine may be ethylenediamine, propylenediamine, hexamethylenediamine, dimer fatty diamines, and homologs thereof. The corresponding cyclohexane derivatives may also be used. In one embodiment, the alkyldiamine may have the formula (tBu)NH—((C2-C20)alkyl)NH(tBu). In another embodiment, the alkyldiamine may have the formula (tBu)NH—((C2-C8)alkyl)NH(tBu).

Suitable trialkanolamines include, but are not limited to, trimethanolamine, triethanolamine, tripropanolamine, triisopropanolamine, tributanolamine, tri-sec-butanolamine, and tri-tert-butanolamine. In one embodiment, the trialkanolamine may be triethanolamine

Suitable condensation reaction catalysts include, but are not limited to, 1,4-diazabicyclo[2.2.2]octane (DABCO, TEDA), dimethylcyclohexylamine (DMCHA), dimethylethanolamine (DMEA), mercury carboxylate, a bismuth compound, such as bismuth octanoate; or tin compound, such as dibutyltin diaceate, dibutyltin dilaurate, dibutyltin dichloride, dibutyltin bis(acetylacetonate), dibutyltin maleate, dibutyltin diisothiocyanate, dibutyltin dimyristate, dibutyltin dioleate, dibutyltin distearate, dibutyltin bis(lauryl mercaptide), dibutyltin bis(isooctylmercaptoacetate), dibutyltin oxide, stannous bis(2-ethylhexoate), stannous oxide, stannous oxatlate, hydrated monobutyltin oxide, monobutyltin trioctoate, dimethyltin salts, and dioctyltin salts. In one embodiment, the condensation reaction catalyst can be dibutyltin diacetate.

In one embodiment, the copolymer can be cured at about room temperature (23° C.) to about 75° C., such as from about 23° C. to about 30° C., from about 30° C. to about 35° C., from about 35° C. to about 40° C., from about 40° C. to about 45° C., from about 45° C. to about 50° C., from about 50° C. to about 55° C., from about 55° C. to about 60° C., from about 60° C. to about 65° C., from about 65° C. to about 70° C., or from about 70° C. to about 75° C. In some embodiments, the copolymer can be cured at a temperature less than 75° C. In some embodiments, the copolymer can be cured at a temperature greater than 23° C.

In one embodiment, the cross-linked poly(urea-urethane) polymer can be a reversible polymer at room temperature. In one embodiment, the stoichiometry of the components can be such that a gel point is achieved. The disclosure also provides a copolymer as described herein in combination with one or more additional polymers. The resulting composition can be, for example, a coating, fiber, adhesive, or plastic. The polyurea or copolymer can be self-healing.

The compounds and compositions can be prepared by any of the applicable techniques of organic synthesis. Many such techniques are well known in the art. Many known techniques are elaborated in Compendium of Organic Synthetic Methods (John Wiley & Sons, New York), Vol. 1, Ian T. Harrison and Shuyen Harrison, 1971; Vol. 2, Ian T. Harrison and Shuyen Harrison, 1974; Vol. 3, Louis S. Hegedus and Leroy Wade, 1977; Vol. 4, Leroy G. Wade, Jr., 1980; Vol. 5, Leroy G. Wade, Jr., 1984; and Vol. 6, Michael B. Smith; as well as standard organic reference texts such as March's Advanced Organic Chemistry: Reactions, Mechanisms, and Structure, 5th Ed. by M. B. Smith and J. March (John Wiley & Sons, New York, 2001), Comprehensive Organic Synthesis; Selectivity, Strategy & Efficiency in Modern Organic Chemistry, in 9 Volumes, Barry M. Trost, Ed.-in-Chief (Pergamon Press, New York, 1993 printing)); Advanced Organic Chemistry, Part B: Reactions and Synthesis, Second Edition, Cary and Sundberg (1983); Protecting Groups in Organic Synthesis, Second Edition, Greene, T. W., and Wutz, P. G. M., John Wiley & Sons, New York; and Comprehensive Organic Transformations, Larock, R. C., Second Edition, John Wiley & Sons, New York (1999).

A number of exemplary methods for preparing the compositions of the disclosure are provided below. These methods are intended to illustrate the nature of such preparations are not intended to limit the scope of applicable methods. Generally, the reaction conditions such as temperature, reaction time, solvents, work-up procedures, and the like, will be those common in the art for the particular reaction. The cited reference material, together with material cited therein, contains detailed descriptions of such conditions. Typically the temperatures will be 100° C. to 200° C., solvents will be aprotic or protic, depending on the conditions and reaction times will be 1 minute to 10 days. Work-up typically consists of quenching any unreacted reagents followed by partition between a water/organic layer system (extraction) and separation of the layer containing the product.

Oxidation and reduction reactions are typically carried out at temperatures near room temperature (about 20° C.), although for metal hydride reductions frequently the temperature is reduced to 0° C. to -100° C. Heating may also be used when appropriate. Solvents are typically aprotic for reductions and may be either protic or aprotic for oxidations. The reaction times are adjusted to achieve desired conversions.

The condensation reactions are typically carried out at temperatures near room temperature, although for non-equilibrating, kinetically controlled condensations reduced temperatures (0° C. to -100° C.) are also common. Solvents can be either protic (common in equilibrating reactions) or aprotic (common in kinetically controlled reactions). Standard synthetic techniques such as azeotropic removal of reaction byproducts and use of anhydrous reaction conditions (e.g. inert gas environments) are common in the art and will be applied when applicable.

### Polymer Characteristics of Keq and Kinetics

To render reversible chemistry dynamic and use the dynamic chemistry for the synthesis of polymers with bulk properties, both the forward and the reverse reaction should be very fast, with large k₁ and k₋₁, and the equilibrium favors the formation of the polymer, large K_{eq}=k₁/k₋₁. In the design of dynamic polyurea specifically, it is thus important to identify a hindered urea bond (HUB) with the properly selected substituent on the amine group so that the corresponding HUB can meet the above. For example, equilibrium and exchange studies using 2-isocyanatoethyl methacrylate and amines with different steric hindrance to identify such HUB have been studied. See for example PCT Publication WO 2014/144539 A2, to The Board of Trustees of the University of Illinois, published Sep. 18, 2014, which is incorporated by reference herein in its entirety.

### EXAMPLES

The following examples further describe and demonstrate embodiments within the scope of the present invention. The Examples are given solely for purpose of illustration and are not to be construed as limitations of the present invention, as many variations thereof are possible without departing from the spirit and scope of the invention.

### Example 1: Shape Memory Polymers

A HUB shape memory polymer was prepared from commercially available monomers: 2-(tert-butylamino)ethanol ( TBAE) and tri-functional homopolymer of hexamethylene diisocyanate (THDI) in the presence of dibutyltin dilaurate (DBTDL) as a catalyst at 60° C. for 12 h. See the following reaction scheme.

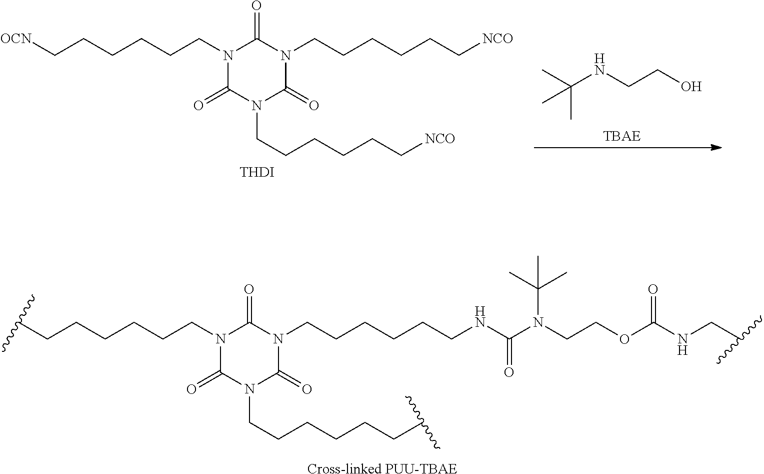

The resulting cross-linked material has a Young's Modulus of ˜2 GPa. Due to the reversible nature of the HUB s, the cross-linked materials are still processible, which could be grounded to powders and molded into shapes such as films or dog bone specimens.

The HUB-SMP has a switchable domain with glass transition temperature at 53° C., which is also the temperature for triggering the shape memory behavior. The HUB-SMP was prepared as a straight band. After heating up to 60° C. (above Tg, the glass transition temperature), the band softened and became elastic. Using external force to deform the band and cooling the sample to room temperature with force applied, the sample could be fixed in a curled shape. Reheating the sample to 60° C. recovers the original shape. When incubating the sample at a high temperature for a long enough time, the dynamic cross-linker can rearrange to reprogram the ‘permanent’ shape of the HUB-SMP. Heating the HUB-SMP at 60° C. for 72 h with an externally applied force reprograms its ‘permanent’ shape from straight to curled. After reprogramming, the HUB-SMP still shows shape memory behavior, but with the opposite shape alternation pattern.

As seen from this example, incorporation of HUB s gives shape memory materials having useful properties. Firstly, the permanent shape of SMPs could be reprogrammed under certain conditions. Secondly, the SMPs are processible although they are covalently cross-linked materials. This means that the permanent shape of SMP could not only be set by curing in a specific mold, but also processed with a variety of other methods such as via a hot press, heat extrusion, or even 3D printing (‘3D printable’ shape memory materials are named as ‘4D printing’ materials).

### Example 2: Malleable, Recyclable, and Healable Thermoset Polymers

A dynamic highly cross-linked poly(urea-urethane) network (PUU-TBAE) containing the corresponding HUB (1-(tert-butyl)-1-ethylurea (TBEU)) with a suitable binding constant (Keq=7.9×10⁵ M⁻¹) and disassociation constants (k⁻¹=0.042 h⁻¹, and 0.21 h⁻¹ at 25° C. and 37° C., respectively) was prepared from commercially available monomers: 2-(tert-butylamino)ethanol (TBAE) and a tri-functional homopolymer of hexamethylene diisocyanate (THDI) in the presence of dibutyltin dilaurate (DBTDL) as a catalyst at 60° C. for 12 h. The polymerization reaction was confirmed by infrared spectroscopy, which revealed that the isocyanate end groups were consumed while urea or urethane bonds were formed. The resulting translucent polymer materials are hard and stiff at room temperature (Tg is ˜53° C.) and have a modulus of 3.5 GPa (analyzed by nanoindenter). Polymeric powders were obtained by grinding the bulk polymer using a pulverization machine.

We then investigated the processability of the PUU-TBAE materials toward complete reprocessing from powder to solid by using a hot press technique. It should be noted that this is a demanding test as the transition from powder to coherent solid requires perfect healing across many thousands of interfaces between particles. Molding a batch of as-synthesized polymer powder under a pressure of 300 kPa for 20 min at 100° C. formed a shaped (film or, dog bone shape, or specific shape) solid polymeric material depending on the type of mold used. The bulk polymeric material after processing is hard and transparent (over 70% transmittance from 400 to 800 nm wavelengths for 200 μm of polymeric film), with a density of 1.04 g/cm3. The polymeric thermoset exhibited a high Young's modulus [E˜3.5 GPa by nanoindendation, 1.9 GPa by dynamic mechanical analysis (DMA)], high hardness (H˜250 MPa) and high breaking strength (˜39.5 MPa). The mechanical properties of this polymer are in the range of commercial, state-of-the-art, cross-linked epoxy resins and unsaturated polyesters.

The bulk materials were subsequently ground into the fine powders and then reprocessed by powder grounding and hot-press molding, which was repeated four additional times. The dynamic mechanical analysis (DMA) results showed that the recycled materials exhibit no major degradation in terms of mechanical strength through five generations of reprocessing as shown in the following table.

Dynamic Mechanical Analysis

| Cycle Number | Modulus (GPa) | Breaking Strength (MPa) |
|---|---|---|
| 1 | 1.87 ± 0.32 | 39.5 ± 9.3 |
| 2 | 1.85 ± 0.36 | 40.0 ± 8.5 |
| 3 | 1.80 ± 0.41 | 40.6 ± 9.2 |
| 4 | 1.71 ± 0.35 | 45.2 ± 8.4 |
| 5 | 1.74 ± 0.37 | 45.0 ± 9.7 |

Overall, this catalyst-free highly cross-linked PUU-TBAE exhibits malleability, indicating that the HUB is resilient against recycling fatigue. As a comparison, the less bulky amine monomers, 2-(iso-propylamino)ethanol (IPAE) and 2-(n-butylamino)ethanol (NBAE) instead of TBAE were used as starting materials for the preparation of PUUs (PUU-IPAE and PUU-NBAE) containing the corresponding HUBs (1-isopropylethylurea (IPEU) and 1-n-butylethylurea (NBEU), respectively) with larger binding constants (Keq>10⁷ M⁻¹) and smaller disassociation constants (k⁻1<0.001 h⁻¹). Both PUU-IPAE and PUU-NBAE exhibited comparable high Young's modulus values. However, both of PUU-IPAE and PUU-NBAE could not be remolded to form shaped materials from powder like materials via hot press because of their low dynamic properties of IPEU and NBEU bonds.

To further understand the mechanism of the malleability in the bulk polymers as a result of HUB exchange under heating conditions, we investigated HUB exchange kinetics at various temperatures in solutions by ¹H NMR spectroscopy. Subsequent to the mixing of two parental model compounds containing TBEU bonds, 1,1′-(ethane-1,2-diyl)bis(1-(tert-butyl)-3-butylurea) (AA), and 1,1′-(ethane-1,2-diyl)bis(3-benzyl-1-(tert-butyl)urea) (BB), the formation of a new TBEU species (AB) was monitored by NMR spectroscopy at three different temperatures: 30° C., 45° C., and 60° C. We observed that the reaction reached equilibrium most quickly at 60° C. while the reaction at 30° C. took the longest time to equilibrate. Though the bond exchange conditions in the bulk polymer would be different from those of small molecules in solution, the model study demonstrates the feasibility of utilizing TBEU exchange reactions as a temperature-dependent approach to achieve malleability of the polymer.

We next tested the self-healing behavior of the PUU-TBAE thermoset. We prepared the dog-bone shaped solid materials with or without Rhodamine 6G staining and made a cut with a razor blade to provide two individual pieces. We then gently brought the two pieces back in contact, and left them at 100 ° C. and 300 kPa pressure environment for 20 min healing without the protection of an inert gas. PUU-TBAE demonstrated the balance of dynamicity, which showed self-healing behavior. The two different colored pieces were healed together. A breaking strain of 95% was recovered within 20 min.

It is seen from this example that a new class of poly(urea-urethane) thermoset (PUU-TBAE) polymers with dynamic covalent hindered urea bonds was developed. This PUU-TBAE thermoset had an excellent malleability which fundamentally behaves like a classic thermoset under ambient conditions yet can be reprocessed by application of heat and pressure. Furthermore, the PUU-TBAE thermoset had a good recyclability which can be recovered from a mixture of traditional thermoplastics and thermosets, and self-healing properties under ambient conditions. These resulting polymers are amenable to low temperature processing conditions and are useful for composites, foamed structures, structural adhesives, coatings, fibers and plastics.

### Example 3: Hydrolysable Polyureas Bearing Hindered Urea Bonds

The references cited in this Example 3 are numbered with respect to this Example 3.

Hydrolysable polymers are widely used materials that have found numerous applications in biomedical, agricultural, plastic and packaging industrials. These polymers usually contain ester and other hydrolysable bonds, such as anhydride, acetal, ketal or imine groups in their backbone structures. Here, we report the design and synthesis of hydrolysable polyureas bearing dynamic hindered urea bonds (HUB s) that can reversibly dissociate to bulky amines and isocyanates, the latter of which can be further hydrolyzed by water, driving the equilibrium to facilitate the degradation of polyureas. Polyureas bearing 1-tert-butyl-1-ethylurea (TBEU) bonds that show high dynamicity (high bond dissociation rates), in the form of either linear polymers or cross-linked gels, can be completely degraded by water under mild conditions. Given the simplicity and low cost for the production of polyureas by simply mixing multifunctional bulky amines and isocyanates, the versatility of the structures and the tunability of the degradation profiles of HUB-bearing polyureas, these materials have potentially very broad applications.

Polymers with transient stability in aqueous solution, also known as hydrolysable polymers, have been applied in many biomedical applications, such as in the design of drug delivery systems,¹ scaffolds for tissue regeneration,² surgical sutures,³ and transient medical devices and implants.⁴ These applications usually require short functioning time, and complete degradation and clearance of materials after their use. Hydrolysable polymers have also been applied in the design of controlled release systems in the agriculture and food industries and used as degradable, environmentally friendly plastics and packaging materials.⁵ Besides polyesters, a class of widely used, conventional hydrolysable materials,⁶ a large variety of other hydrolysable polymers bearing anhydride,⁷ orthoester,⁸ acetal,⁹ ketal,¹⁰ aminal,¹¹ hemiaminal,¹¹⁻¹² imine,¹³ phosphoester,¹⁴ and phosphazene¹⁵ groups have also been reported. Syntheses of these polymers usually involves condensation^{2d} or ring-opening polymerization,¹⁶ and these syntheses typically involve removal of byproducts^{2d} and employ high reaction temperature^{2d} and/or metal catalysts,^{6b} which complicates the material preparation. In this study, we report the design of polyureas bearing hindered urea bonds (HUB s) as potentially one of the least expensive degradable polymers that can be easily synthesized by mixing multifunctional bulky amines and isocyanates, expanding the family of hydrolysable polymers.

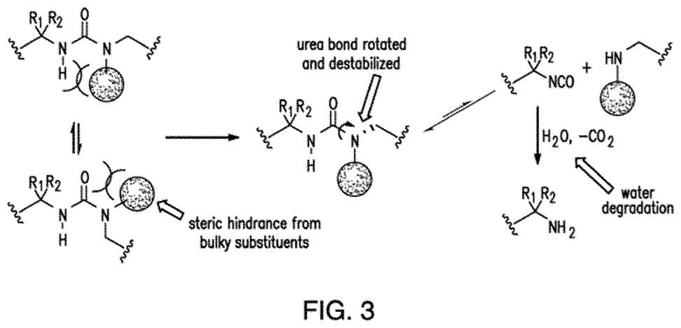

Polyureas are commonly used as fiber, coating and adhesive materials. They can be readily synthesized via addition reaction of widely available, di- or multifunctional isocyanates and amines that do not require the use of catalysts and extreme reaction conditions and do not produce any byproducts. Urea groups are one of the most stable chemical bonds against further reactions including hydrolysis due to the conjugation stabilization effects of its dual amide structure. However, urea bonds can be destabilized by incorporating bulky substituents to one of the nitrogen atoms, by means of disturbing the orbital co-planarity of the amide bonds that diminishes the conjugation effect (FIG. 3).¹⁷ Urea bonds bearing a bulky substituent, or hindered urea bonds (HUBs), can reversibly dissociate into isocyanate and amines and show interesting dynamic property. The fast reversible reactions between HUBs and isocyanates/amines have been the basis in our recent design of self-healing polyureas.¹⁸ Because isocyanates can be subject to hydrolysis in aqueous solution to form amines and carbon dioxide, an irreversible process that shifts the equilibrium to favor the HUB dissociation reaction and eventually lead to irreversible and complete degradation of HUBs (FIG. 3), we reason that HUBs can be used to design easily available hydrolysable polymers potentially for the numerous applications abovementioned. Herein, we report the development of HUB-based polyureas that can be hydrolyzed with hydrolytic degradation kinetics tunable by the steric hindrance of the HUB structures.

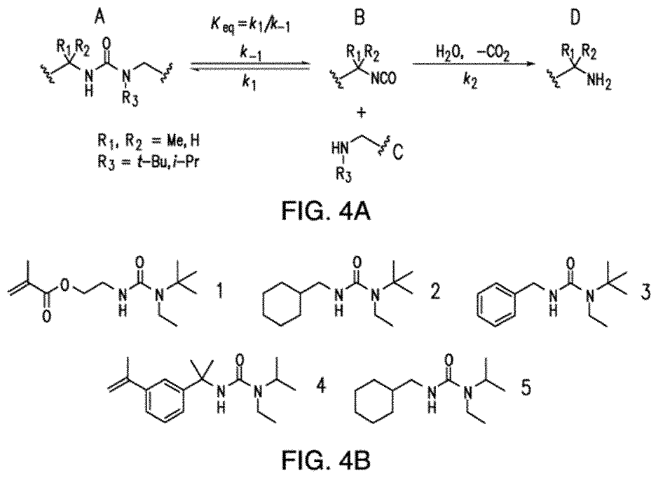

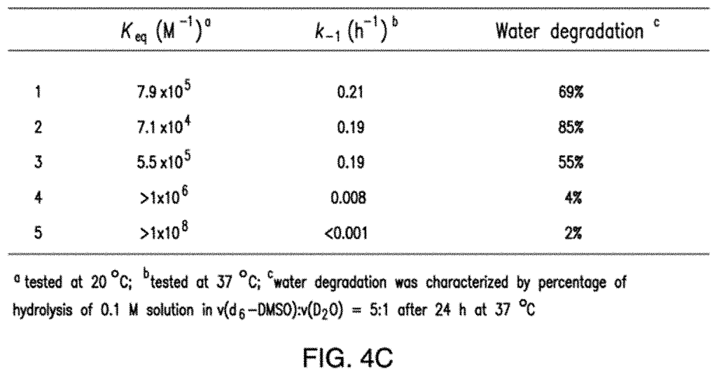

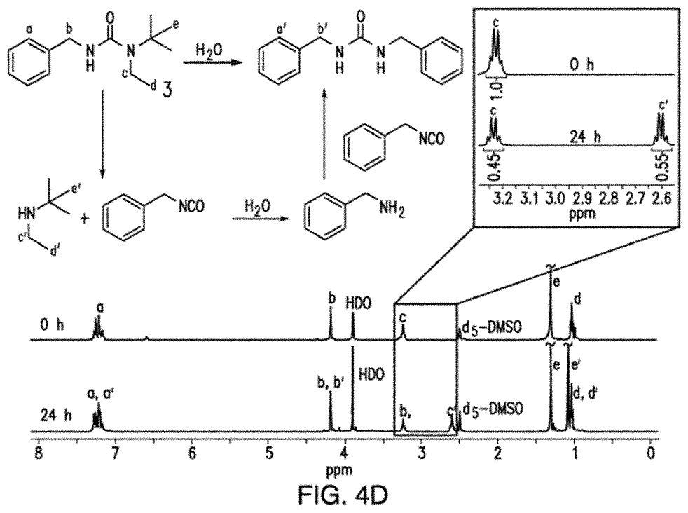

The property of a dynamic covalent bond can be expressed by its K_{eq}, the binding constant showing the thermodynamic stability of the dynamic bond, and its k₋₁, the dissociation rate of the dynamic bond. According to the hydrolytic degradation mechanism of a HUB shown in FIG. 4A, the rate of hydrolysis equals to the rate of the formation of product D, which can be expressed by Equation (1):

$$r ( hydrolysis ) = \frac{d [ D ]}{dt} = {k}_{2} [ B ] [ {H}_{2} O ] \qquad ( 1 )$$

Since the isocyanate B is a dissociative intermediate with very low concentration, a steady-state approximation expressed as Equation (2) is thus deduced:

k₂[B] [H₂O]+k₁[B] [C]=k₋₁[A] (2)

As K_{eq}=k₁/k₋₁, Equation (3) can thus be deduced from Equation (1) and (2):

$$r ( hydrolysis ) = \frac{{k}_{2} [ A ] [ {H}_{2} O ]}{{K}_{eq} [ C ] + \frac{{k}_{2}}{{k}_{- 1}} [ {H}_{2} O ]} \qquad ( 2 )$$

According to Equation 3, the hydrolysis kinetics is related to both K_{eq} and k₋₁, with smaller K_{eq} and larger k₋₁ giving faster hydrolysis. This is consistent with the notion that more dynamic HUBs (more bulky N-substituents) give faster hydrolytic degradation. To confirm this, we analyzed the dynamic parameters¹⁸ and the hydrolysis kinetics of five different HUB-containing model compounds (1-5, See FIG. 4B) with their dynamicity and hydrolytic degradation parameters summarized in FIG. 4C. All five compounds were synthesized by mixing the corresponding isocyanates and amines at 1:1 molar ratio. Compounds 1-3 have similar bulkiness, which are all based on 1,1-tert-butylethylurea (TBEU, R₃=tent-butyl) structure. They show nearly identical k₋₁. Compounds 4 and 5 have less bulky 1-iso-propyl-1-ethylurea (IPEU, R₃=iso-propyl) structure, which show lower dynamicity than 1-3 (higher K_{eq} and lower k₁). For these two IPEU based compounds, 4 shows higher dynamicity than 5 with lower K_{eq} and higher k₋₁ due to its more bulky isocyanate structure (more bulky R₁ and R₂).

We went on to analyze the hydrolytic degradation profiles of 1-5 by ¹H NMR. The compounds were dissolved in a mixture of d₆-DMSO and D₂O (v(d₆-DMSO)/v(D₂O)=5:1). The percentage of the hydrolyzed products was analyzed after the mixture was incubated for 24 h at 37° C. (See FIG. 4D; the hydrolytic degradation of 3 is shown as an example). All three TBEU based compounds (1-3) showed over 50% of hydrolytic degradation of their urea bonds, with 2 showing the fastest degradation (85%) due to its lowest K_{eq}. Compound 4, bearing less bulky (less dynamic) IPEU structure, showed slower hydrolytic degradation (˜10%) compared to 1-3. No detectable hydrolysis was observed for compound 5 because of its least substituent bulkiness (lowest dynamicity, FIG. 4C). These results are consistent with the conclusions drawn from Equation 3.

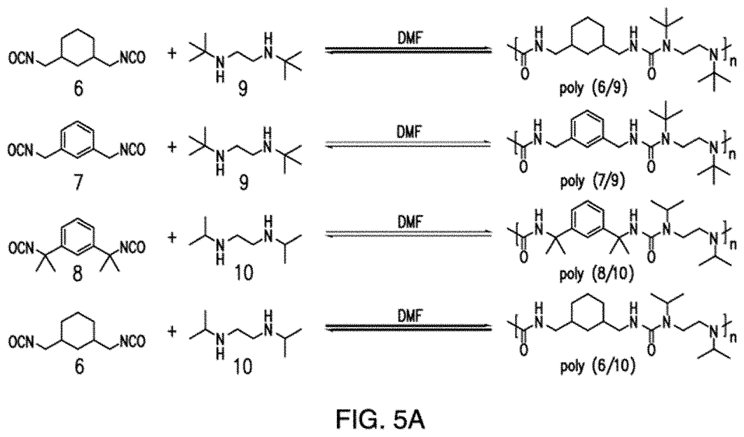

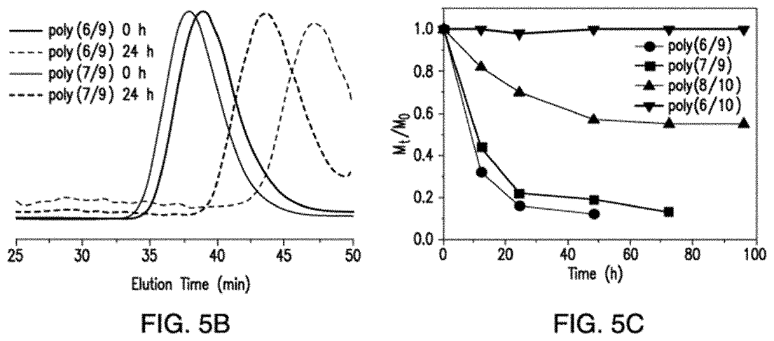

We next examined if polymers bearing HUBs (pHUBs) could also be degraded by water. Linear pHUBs were synthesized by mixing diisocyanates and diamines at a 1:1 molar ratio in DMF. Although the bulky substituents in HUBs destabilize the urea bond, the HUBs still have sufficiently large binding constants (K_{eq}˜10⁵, see FIG. 4C) to form high molecular weight polymers. Poly(6/9), poly(7/9), poly(8/10), and poly(6/10), four different pHUBs with descending dynamicity, were prepared by mixing the corresponding diisocyanate (1,3-bis(isocyanatomethyl)cyclohexane (6), 1,3-bis(isocyanatomethyl)benzene (7) or 1,3-bis(1-isocyanato-1-methylethyl)benzene (8)) and diamine (N,N′-di-tert-butylethylenediamine (9) or N,N′-di-iso-propylethylenediamine (10)). The HUB structure of poly(6/9), poly(7/9), poly(8/10) and poly(6/10) resembles the corresponding model compounds 2-5 (FIG. 5A). The Mₙ's of these four polymers were 22, 22, 44 and 120 KDa, as characterized by gel permeation chromatography (GPC), and showed correlation with their K_{eq}'s. To study the hydrolytic degradation of these pHUBs, 5% of water was added to the DMF solutions of each polymer. These solutions were vigorously stirred and incubated at 37° C., and the molecular weights were monitored by GPC at selected time. MW decrease was observed for TBEU based poly(6/9) and poly(7/9) (FIG. 5B). For IPEU based polymers, poly(8/10) showed limited degradation, while poly(6/10) barely showed any change of its Mₙ after 24 h (FIG. 5C). After incubation for 48 h, the percentages of MW reduction for poly(6/9), poly(7/9) and poly(8/10) were 88%, 81% and 43%, respectively. The MW of poly(8/10) did not further decrease for elongated incubation (FIG. 5C), which could be attributed to the increase of free amine concentration that inhibits degradation (see Equation 3, larger [C] gives lower degradation rate). The alteration of polymer hydrolysis kinetics with the change of HUB bulkiness was consistent with the results derived from the study of small molecular model compounds 1-5.

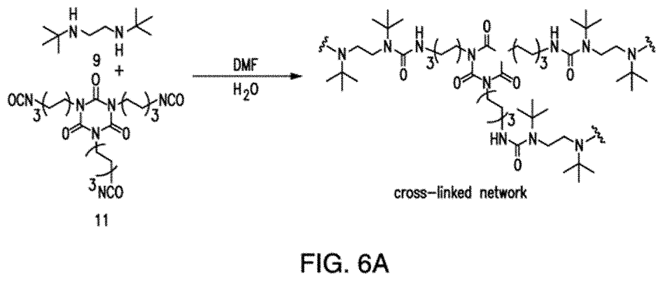

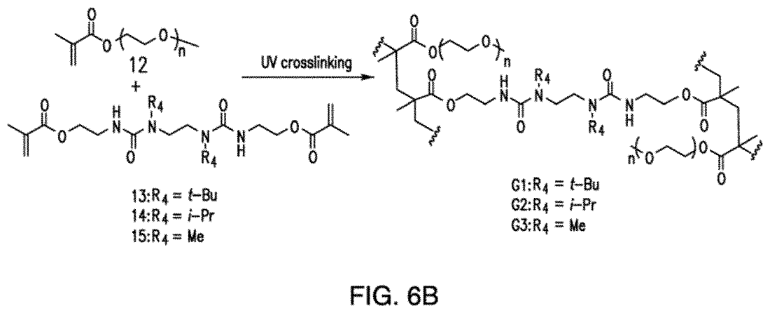

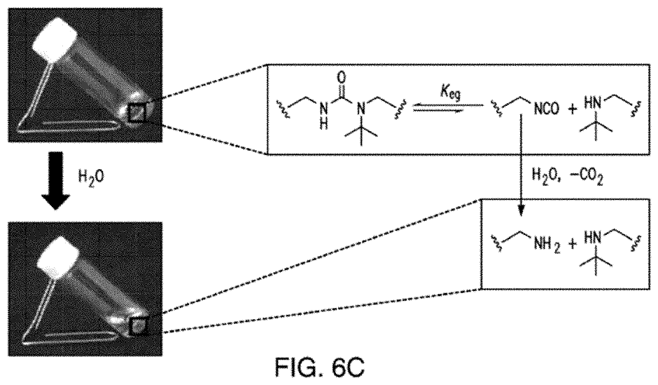

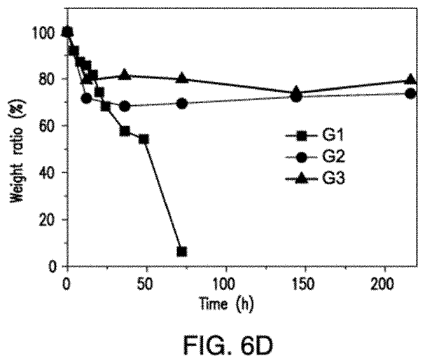

To further demonstrate the hydrolytic degradation of TBEU based polymer, we prepared a cross-linked organogel by mixing tri-isocyanate 11 with diamine 9 in DMF containing 5% water. Because isocyanate reacts with amine much faster than with water, 9 and 11 first reacted to form polyurea gel. The added water slowly hydrolyzed the TBEU bond, which led to the collapse of the gel after the gel was incubated 24 h at 37° C. (FIGS. 6A and 6C).

To study pHUBs degradation in aqueous solution and explore the potential of pHUBs for biomaterials applications, we designed hydrophilic polymers bearing HUB cross-linkers. To poly(ethylene glycol) methyl ether methacrylate monomer (Mₙ˜500), we added HUB containing dimethacrylate 13-14 as cross-linkers and Irgacure 2959 as the photoinitiator. The HUB s structures in 13-14 are TBEU and IPEU, respectively. The mixtures were irradiated by UV light (365 nm) to prepare the cross-linked polymers G1, G2, and G3. (FIG. 6B). We first did dynamic exchange studies of G1, G2, and G3 by immersing them in DMF in the presence or absence of hexylamine. In the absence of hexylamine, all three gels swelled, demonstrating they are cross-linked polymers. In the presence of hexylamine, only G1 was dissolved while G2 and G3 stayed intact. This experiment demonstrated that TBEU-containing G1 has much faster dynamic exchange than G2 or G3, which is the requisite for efficient water degradation. For the water degradation study, we immersed G1, G2 and G3 into phosphate buffered saline (PBS) and monitored the weight change at various time with the incubation at 37° C. (gels were pre-treated with deionized water with short time to remove all the unreacted monomers).^{2d} The weights of G2 and G3 remained nearly unchanged after incubation for 9 days. In contrast, G1 showed a consistent weight decrease and completely disappeared after incubation for 4 days (FIG. 6B). We should notice that the degradation of TBEU might give a stable urea as the product since the amine from hydrolysis of isocyanate might react with another isocyanate molecule (as shown in the example in FIG. 4D), which will hold the network without complete degradation. However, we observed complete degradation of G1 in PBS, which meant that the formation of stable urea rarely happened in this case. Several reasons might explain the reduced probability of urea coupling: i) much higher water concentration in pure water environment than organic solvent environment; ii) protonation of amine groups in buffered neutral pH reduces reactivity; iii) amine groups are embedded by long oligo-ethylene glycol chains, which block their reaction of the exposed isocyanate.

In conclusion, we demonstrated the potential of HUBs for the design of water degradable polymeric materials. Kinetic analyses of small molecule model compounds prove that more bulky HUB s lead to faster water degradations. The same trend applies to the polymeric materials, with TBEU as one of the HUBs having the appropriate bulkiness for both sufficient binding stability for polymer formation and efficient dynamicity for water degradation. TBEU based linear polymers degrades to 10%-20% of their original size within 2 days. TBEU is also incorporated into cross-linked hydrogel materials which render complete water dissolution of the hydrogel within 4 days, making pHUBs alternative building blocks of hydrolysable hydrogels. pHUBs provide a great new platform for the engineering of hydrolysable materials. Firstly, the degradation kinetics could be directly controlled by substituents bulkiness. While we have demonstrated the use of TBEU for water degradable materials within days under mild conditions, less bulky urea might be used for applications which need longer lasting time or harsher degradation conditions (such as poly(8/10) or its derivatives). Secondly, different from traditional hydrolysable polymers, pHUBs could be synthesized by simple mixing amine and isocyanate precursors at ambient condition with no catalyst and with no further purification needed, and with no byproducts generated, which makes it possible for end-users to control the copolymer composition for specific uses without the need of a complicated synthetic apparatus. Additionally, a large number of isocyanate monomers have been developed for use in the polyurethane and polyurea plastic industry, which can be used to react with amines with N-bulky substituents to give a very large library of hydrolysable polymers with versatile structures and functions.

### REFERENCES FOR EXAMPLE 3

(1) (a) Petros, R. A.; DeSimone, J. M. Nat. Rev. Drug Discov. 2010, 9, 615-627. (b) Duncan, R. Nat. Rev. Cancer 2006, 6, 688-701. (c) Tong, R.; Cheng, J. J. Polymer Reviews 2007, 47, 345-381. (d) Yin, Q.; Tong, R.; Yin, L. C.; Fan, T. M.; Cheng, J. J. Polym. Chem. 2014, 5, 1581-1585.

(2) (a) Langer, R.; Vacanti, J. P. Science 1993, 260, 920-926. (b) Sun, H. L.; Meng, F. H.; Dias, A. A.; Hendriks, M.; Feijen, J.; Zhong, Z. Y. Biomacromolecules 2011, 12, 1937-1955. (c) Annabi, N.; Tamayol, A.; Uquillas, J. A.; Akbari, M.; Bertassoni, L. E.; Cha, C.; Camci-Unal, G.; Dokmeci, M. R.; Peppas, N. A.; Khademhosseini, A. Adv. Mater. 2014, 26, 85-124. (d) Wang, Y. D.; Ameer, G. A.; Sheppard, B. J.; Langer, R. Nat. Biotechnol. 2002, 20, 602-606.

(3) (a) Ulery, B. D.; Nair, L. S.; Laurencin, C. T. J. Polym. Sci., Part B: Polym. Phys. 2011, 49, 832-864. (b) Lendlein, A.; Langer, R. Science 2002, 296, 1673-1676

(4) (a) Hwang, S. W.; Tao, H.; Kim, D. H.; Cheng, H. Y.; Song, J. K.; Rill, E.; Brenckle, M. A.; Panilaitis, B.; Won, S. M.; Kim, Y. S.; Song, Y. M.; Yu, K. J.; Ameen, A.; Li, R.; Su, Y. W.; Yang, M. M.; Kaplan, D. L.; Zakin, M. R.; Slepian, M. J.; Huang, Y. G.; Omenetto, F. G.; Rogers, J. A. Science 2012, 337, 1640-1644. (b) Hwang, S. W.; Park, G.; Cheng, H.; Song, J. K.; Kang, S. K.; Yin, L.; Kim, J. H.; Omenetto, F. G.; Huang, Y. G.; Lee, K. M.; Rogers, J. A. Adv. Mater. 2014, 26, 1992-2000.

(5) (a) Gross, R. A.; Kalra, B. Science 2002, 297, 803-807. (b) Tharanathan, R. N. Trends Food Sci. Technol. 2003, 14, 71-78.

(6) (a) Nampoothiri, K. M.; Nair, N. R.; John, R. P. Bioresour. Technol. 2010, 101, 8493-8501. (b) Tong, R.; Cheng, J. J. Angew. Chem. Int. Ed. 2008, 47, 4830-4834. (c) Yoshimoto, H.; Shin, Y. M.; Terai, H.; Vacanti, J. P. Biomaterials 2003, 24, 2077-2082. (d) Samarajeewa, S.; Shrestha, R.; Li, Y. L.; Wooley, K. L. J. Am. Chem. Soc. 2012, 134, 1235-1242. (e) Almutairi, A.; Rossin, R.; Shokeen, M.; Hagooly, A.; Ananth, A.; Capoccia, B.; Guillaudeu, S.; Abendschein, D.; Anderson, C. J.; Welch, M. J.; Frechet, J. M. J. Proc. Natl. Acad. Sci. USA 2009, 106, 685-690

(7) (a) Yin, L.; Huang, X.; Xu, H. X.; Zhang, Y. F.; Lam, J.; Cheng, J. J.; Rogers, J. A. Adv. Mater. 2014, 26, 3879-3884. (b) Rutherglen, B. G.; McBath, R. A.; Huang, Y. L.; Shipp, D. A. Macromolecules 2010, 43, 10297-10303.

(8) (a) Heller, J.; Barr, J.; Ng, S. Y.; Abdellauoi, K. S.; Gurny, R. Adv. Drug Del. Rev. 2002, 54, 1015-1039. (b) Du, F. S.; Huang, X. N.; Chen, G. T.; Lin, S. S.; Liang, D. H.; Li, Z. C. Macromolecules 2010, 43, 2474-2483.

(9) (a) Binauld, S.; Stenzel, M. H. Chem. Commun. 2013, 49, 2082-2102. (b) Liu, R.; Zhang, Y.; Zhao, X.; Agarwal, A.; Mueller, L. J.; Feng, P. Y. J. Am. Chem. Soc. 2010, 132, 1500-1501.

(10) (a) Shenoi, R. A.; Narayanannair, J. K.; Hamilton, J. L.; Lai, B. F. L.; Horte, S.; Kainthan, R. K.; Varghese, J. P.; Rajeev, K. G.; Manoharan, M.; Kizhakkedathu, J. N. J. Am. Chem. Soc. 2012, 134, 14945-14957. (b) Zhang, Y. F.; Wang, R.; Hua, Y. Y.; Baumgartner, R.; Cheng, J. J. ACS Macro Lett. 2014, 3, 693-697. (c) Broaders, K. E.; Pastine, S. J.; Grandhe, S.; Frechet, J. M. J. Chem. Commun. 2011, 47, 665-667.

(11) Garcia, J. M.; Jones, G. O.; Virwani, K.; McCloskey, B. D.; Boday, D. J.; ter Huurne, G. M.; Horn, H. W.; Coady, D. J.; Bintaleb, A. M.; Alabdulrahman, A. M. S.; Alsewailem, F.; Almegren, H. A. A.; Hedrick, J. L. Science 2014, 344, 732-735.

(12) Fishman, J. M.; Kiessling, L. L. Angew. Chem. Int. Ed. 2013, 52, 5061-5064

(13) (a) Kim, Y. H.; Park, J. H.; Lee, M.; Kim, Y. H.; Park, T. G.; Kim, S. W. J. Controlled Release 2005, 103, 209-219. (b) Zhao, Y. L.; Li, Z. X.; Kabehie, S.; Botros, Y. Y.; Stoddart, J. F.; Zink, J. I. J. Am. Chem. Soc. 2010, 132, 13016-13025. (c) Xu, X. W.; Flores, J. D.; McCormick, C. L. Macromolecules 2011, 44, 1327-1334.

(14) (a) Xiong, M. H.; Bao, Y.; Yang, X. Z.; Wang, Y. C.; Sun, B. L.; Wang, J. J. Am. Chem. Soc. 2012, 134, 4355-4362. (b) Zhang, S. Y.; Zou, J.; Zhang, F. W.; Elsabahy, M.; Felder, S. E.; Zhu, J. H.; Pochan, D. J.; Wooley, K. L. J. Am. Chem. Soc. 2012, 134, 18467-18474.

(15) (a) Deng, M.; Kumbar, S. G.; Nair, L. S.; Weikel, A. L.; Allcock, H. R.; Laurencin, C. T. Adv. Funct. Mater. 2011, 21, 2641-2651. (b) Park, M. R.; Chun, C. J.; Ahn, S. W.; Ki, M. H.; Cho, C. S.; Song, S. C. J. Controlled Release 2010, 147, 359-367.

(16) Ajellal, N.; Carpentier, J. F.; Guillaume, C.; Guillaume, S. M.; Helou, M.; Poirier, V.; Sarazin, Y.; Trifonov, A. Dalton Trans. 2010, 39, 8363-8376.

(17) (a) Grover, A. K.; Kapoor, M. Can. J. Biochem. 1973, 51, 363-378. (b) Hutchby, M.; Houlden, C. E.; Ford, J. G.; Tyler, S. N. G.; Gagne, M. R.; Lloyd-Jones, G. C.; Booker-Milburn, K. I. Angew. Chem. Int. Ed. 2009, 48, 8721-8724

(18) Ying, H. Z.; Zhang, Y. F.; Cheng, J. J. Nat. Commun. 2014, 5:3218.

### INCORPORATION BY REFERENCE

The entire disclosure of each of the patent documents, including certificates of correction, patent application documents, scientific articles, governmental reports, websites, and other references referred to herein is incorporated by reference herein in its entirety for all purposes. In case of a conflict in terminology, the present specification controls.

### EQUIVALENTS

The invention can be embodied in other specific forms without departing from the spirit or essential characteristics thereof. The foregoing embodiments are to be considered in all respects illustrative rather than limiting on the invention described herein. In the various embodiments of the methods and systems of the present invention, where the term comprises is used with respect to the recited steps or components, it is also contemplated that the methods and systems consist essentially of, or consist of, the recited steps or components. Further, it should be understood that the order of steps or order for performing certain actions is immaterial so long as the invention remains operable. Moreover, two or more steps or actions can be conducted simultaneously.

In the specification, the singular forms also include the plural forms, unless the context clearly dictates otherwise. Unless defined otherwise, all technical and scientific terms used herein have the same meaning as commonly understood by one of ordinary skill in the art to which this invention belongs. In the case of conflict, the present specification will control.

All percentages and ratios used herein, unless otherwise indicated, are by weight. Also, throughout the disclosure the term “weight” is used. It is recognized the mass of an object is often referred to as its weight in everyday usage and for most common scientific purposes, but that mass technically refers to the amount of matter of an object, whereas weight refers to the force experienced by an object due to gravity. Also, in common usage the “weight” (mass) of an object is what one determines when one “weighs” (masses) an object on a scale or balance.

What is claimed is:

1. A hindered urea bond polymer comprising recurring units from:
(a) an unsaturated monomer substituted by a hindered amine, and
(b) a crosslinking agent comprising two or more isocyanate groups.

2. The polymer according to claim 1 wherein the unsaturated monomer substituted by a hindered amine is selected from the group consisting of acrylates, butadienes, ethylenes, styrenes, vinyl esters, vinyl ethers, and combinations thereof.

3. A polymer according to claim 2 wherein the unsaturated monomer substituted by a hindered amine is selected from 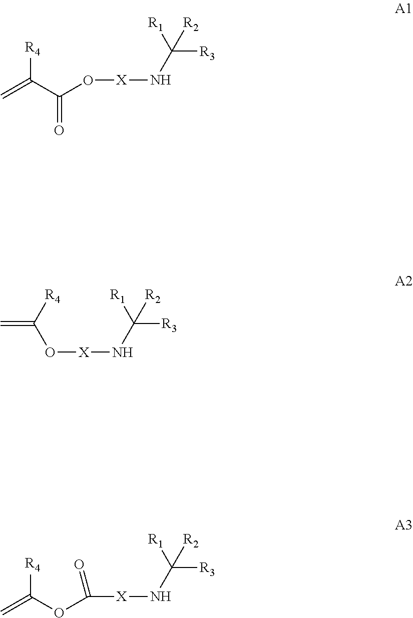
wherein R₁ is selected from the group consisting of (C₁-C₂₀)alkyl, (C₄-C₁₀)cyclolalkyl, (C₁-C₂₀)alkyl(C₄-C₁₀)cycloalkyl, (C₁-C₂₀)alkyl(C₄-C₁₀)cycloalkyl(C₁-C₂₀)alkyl, (C₁-C₂₀)alkyl(C₆-C₁₀)aryl(C₁-C₂₀)alkyl, (C₂-C₂₀)alkyl-PolyEthyleneGlycol-(C₂-C₂₀)alkyl,
R₂, R₃, and R₄ are independently selected from the group consisting of (C₁-C₂₀)alkyl, (C₄-C₁₀)cyclolalkyl, (C₁-C₂₀)alkyl(C₄-C₁₀)cycloalkyl, (C₁-C₂₀)alkyl(C₄-C₁₀)cycloalkyl(C₁-C₂₀)alkyl, (C₁-C₂₀)alkyl(C₆-C₁₀)aryl(C₁-C₂₀)alkyl, (C₂-C₂₀)alkyl-PEG-(C₂-C₂₀)alkyl, and H;
and X is independently selected from a single bond, (C₁-C₂₀)alkyl, (C₄-C₁₀)cyclolalkyl, (C₁-C₂₀)alkyl(C₄-C₁₀)cycloalkyl, (C₁-C₂₀)alkyl(C₄-C₁₀)cycloalkyl(C₁-C₂₀)alkyl, (C₁-C₂₀)alkyl(C₆-C₁₀)aryl(C₁-C₂₀)alkyl, and (C₂-C₂₀)alkyl-PEG-(C₂-C₂₀)alkyl, and combinations thereof.

4. The polymer according to claim 3 wherein R₄ is H or methyl.

5. The polymer according to claim 4 wherein the unsaturated monomer substituted by a hindered amine is a compound of formula Al.

6. The polymer according to claim 5 wherein R₁, R₂, R₃, are each methyl.

7. The polymer according claim 1 wherein the crosslinking agent is OCN—Y—NCO, where Y is selected from (C₂-C₂₀)alkyl, (C₄-C₁₀)cyclolalkyl, (C₁-C₂₀)alkyl(C₄-C₁₀)cycloalkyl, (C₁-C₂₀)alkyl(C₄-C₁₀)cycloalkyl(C₁-C₂₀)alkyl, (C₁-C₂₀)alkyl(C₆-C₁₀)aryl(C₁-C₂₀)alkyl, and (C₂-C₂₀)alkyl-PolyEthyleneGlycol-(C₂-C₂₀)alkyl, and combinations thereof.

8. A hindered urea bond polymer comprising recurring units from:
(a) an unsaturated monomer substituted by an isocyanate group, and
(b) a crosslinking agent substituted with two or more hindered amine groups.

9. The polymer according to claim 9 wherein the unsaturated monomer substituted by an isocyanate group is selected from the group of acrylates, methacrylates, butadienes, ethylenes, norbornenes, styrenes, vinyl chlorides, vinyl esters, vinyl ethers, and combinations thereof.

10. The polymer according to claim 9 wherein the isocyanate-substituted monomer is selected from 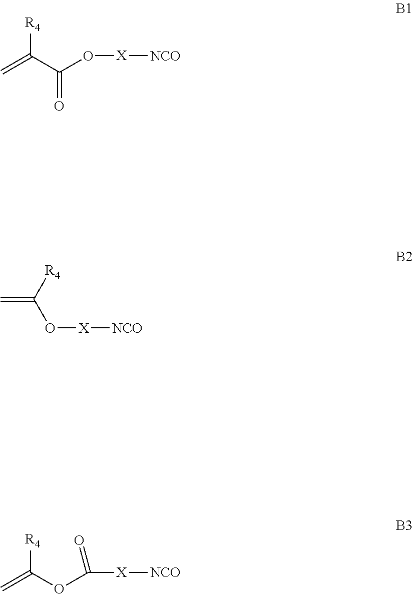
wherein R4 is selected from the group consisting of (C₁-C₂₀)alkyl, (C₄-C₁₀)cyclolalkyl, (C₁-C₂₀)alkyl(C₁-C₁₀)cycloalkyl, (C₁-C₂₀)alkyl(C₁-C₁₀)cycloalkyl(C₁-C₂₀)alkyl, (C₁-C₂₀)alkyl(C₆-C₁₀)aryl(C₂-C₂₀)alkyl-PolyEthyleneGlycol-(C₂-C₂₀)alkyl, and H; and
X is a single bond, (C₁-C₂₀)alkyl, (C₄-C₁₀)cyclolalkyl, (C₁-C₂₀)alkyl(C₄-C₁₀)cycloalkyl, (C₁-C₂₀)alkyl(C₄-C₁₀)cycloalkyl(C₁-C₂₀)alkyl, (C₁-C₂₀)alkyl(C₆-C₁₀)aryl(C₁-C₂₀)alkyl, and (C₂-C₂₀)alkyl-PolyEthyleneGlycol-(C₂-C₂₀)alkyl, and combinations thereof.

11. The polymer according to claim 10 wherein the unsaturated monomer substituted by an isocyanate comprises a compound of formula B1 wherein R₄ is H or methyl.

12. A polymer according to claim 9 wherein the crosslinking agent is 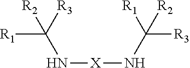
wherein R₁, R₂, and R₃, are independently selected from the group consisting of (C₁-C₂₀)alkyl, (C₄-C₁₀)cyclolalkyl, (C₁-C₂₀)alkyl(C₄-C₁₀)cycloalkyl, (C₁-C₂₀)alkyl(C₄-C₁₀)cycloalkyl(C₁-C₂₀)alkyl, (C₁-C₂₀)alkyl(C₆-C₁₀)aryl(C₁-C₂₀)alkyl, (C₂-C₂₀)alkyl-PolyEthyleneGlycol-(C₂-C₂₀)alkyl, and H; and
X is selected from (C₂-C₂₀)alkyl, (C₄-C₁₀)cyclolalkyl, (C₁-C₂₀)alkyl(C₄-C₁₀)cycloalkyl, (C₁-C₂₀)alkyl(C₄-C₁₀)cycloalkyl(C₁-C₂₀)alkyl, (C₁-C₂₀)alkyl(C₆-C₁₀)aryl(C₁-C₂₀)alkyl, and (C₂-C₂₀)alkyl-PolyEthyleneGlycol-(C₂-C₂₀)alkyl, and combinations thereof.

13. The polymer according to claim 12 wherein R₁ and R₂ are selected from the group consisting of (C₁-C₂₀)alkyl, (C₄-C₁₀)cyclolalkyl, (C₁-C₂₀)alkyl(C₄-C₁₀)cycloalkyl, (C₁-C₂₀)alkyl(C₄-C₁₀)cycloalkyl(C₁-C₂₀)alkyl, (C₁-C₂₀)alkyl(C₆-C₁₀)aryl(C₁-C₂₀)alkyl, (C₂-C₂₀)alkyl-PolyEthyleneGlycol-(C₂-C₂₀)alkyl; and
R₃ is selected from the group consisting of (C₁-C₂₀)alkyl, (C₄-C₁₀)cyclolalkyl, (C₁-C₂₀)alkyl(C₄-C₁₀)cycloalkyl, (C₁-C₂₀)alkyl(C₄-C₁₀)cycloalkyl(C₁-C₂₀)alkyl, (C₁-C₂₀)alkyl(C₆-C₁₀)aryl(C₁-C₂₀)alkyl, (C₂-C₂₀)alkyl-PolyEthyleneGlycol-(C₂-C₂₀)alkyl, and H.

14. A polymer according to claim 12 wherein R₁, R₂ and R₃ are each methyl.

15. The hindered urea bond polymer according to claim 2 comprising recurring units from:
(c) an additional monomer selected from acrylates, butadienes, ethylenes, styrenes, vinyl esters, vinyl ethers, and combinations thereof.

16. The hindered urea bond polymer according to claim 15 wherein the additional monomer comprises an acrylate or methacrylate.

17. The hindered urea bond polymer according to claim 9 comprising recurring units from:
(c) an additional monomer selected from acrylates, butadienes, ethylenes, styrenes, vinyl esters, vinyl ethers, and combinations thereof.

18. The hindered urea bond polymer according to claim 17 wherein the additional monomer comprises an acrylate or methacrylate.

18. The hindered urea bond polymer according to claim 12 comprising recurring units from:
(c) an additional monomer selected from acrylates, butadienes, ethylenes, styrenes, vinyl esters, vinyl ethers, and combinations thereof.

19. The hindered urea bond polymer according to claim 18 wherein the additional monomer comprises an acrylate or methacrylate.

20. A biodegradable packaging material, drug delivery system, medical device, surgical suture or scaffold for tissue regeneration comprising a polymer according to claim 2.

21. A biodegradable packaging material, drug delivery system, medical device, surgical suture or scaffold for tissue regeneration comprising a polymer according to claim 9.
